(19) United States
(12) Patent Application Publication
(10) Pub. No.: US 2007/0141101 A1
(43) Pub. Date: Jun. 21, 2007

(54) Method for stimulating angiogenesis and wound healing

(75) Inventors: Matthew A. Nugent, Bedford, MA (US); Adrienne L. Georges, Somerville, MA (US)

Correspondence Address: RONALD I. EISENSTEIN, 100 SUMMER STREET, NIXON PEABODY LLP, BOSTON, MA, 02110, US

(73) Assignee: The Trustees of Boston University, Boston, MA (US)

(21) Appl. No.: 10/552,194

(22) PCT Filed: Apr. 12, 2004
(86) PCT No.: PCT/US04/11168

§ 371 Date: Oct. 12, 2006

Related U.S. Application Data
(60) Provisional application No. 60/461,974, filed on Apr. 10, 2003.

(51) Int. Cl. A61F 2/00; A61K 38/16

(52) U.S. Cl. 424/423; 514/8

(57) ABSTRACT

A device comprising an extracellular matrix having an internal pH between 4.0 and 6.0 is discussed. This matrix contains heparin or a heparin-related compound such as heparan sulfate. Preferably the matrix also contains fibronectin or a fragment thereof. The matrix will bind to a protein having a pH dependent binding to heparin such as VEGF, preferably VEGF 121 or VEGF 165. The device will release the protein as the pH increases to physiological pH, such as 7.0 to 7.5. The device can be used to deliver a drug to a specific site. For example, with VEGF to a site in need of angiogenesis.

This invention was supported by National Institutes of Health grant HL56200 and the government of the United States has certain rights thereto.

### FIELD OF THE INVENTION

The present application is directed to a method of delivering a protein bound to an extracellular matrix under acidic conditions that is released upon exposure to physiological pH and a device comprising such an extracellular matrix. Preferably such an extracellular matrix bound protein can be used in stimulating angiogenesis and for example, enhancing wound healing, where the bound protein is an angiogenic promoter, preferably VEGF, that is released upon exposure to physiological pH.

### BACKGROUND OF THE INVENTION

Angiogenesis, the growth of new blood vessels, is a complex process involving the disruption of vascular basement membranes, migration and proliferation of endothelial cells, and subsequent blood vessel formation and maturation. Several mediators are known to elicit angiogenic responses, and administration of these mediators promotes revascularization of ischemic tissues. Vascular endothelial growth factor (VEGF protein) is one of the most specific of the known angiogenic mediators due to localization of its receptors almost exclusively on endothelial cells. Receptors for VEGF are upregulated under ischemic conditions, and the administration of recombinant VEGF augments the development of collateral vessels and improves function in peripheral and myocardial ischemic tissue.

The delivery of such angiogenic promoters such as VEGF protein remains a significant challenge. For example, the half-life of VEGF protein is very short; the administration of high doses of VEGF protein is associated with hypotension, and systemic administration of VEGF protein can cause promiscuous induction of angiogenesis in tissues other than that which has been targeted. Promiscuous induction of angiogenesis can cause blindness, increase the aggressiveness of tumor cells, and lead to a multitude of other negative side-effects. Furthermore, the quantity of VEGF protein delivered is important. If too little VEGF protein is delivered, angiogenesis will not be induced, and a significant therapeutic benefit will not be achieved. If too much VEGF protein is delivered, the formation of disorganized vasculature beds, loss of function in the affected tissue, and promiscuous angiogenesis can result.

Attempts to address these problems have utilized methods wherein multiple applications of an angiogenic mediator were made to a target tissue. For example, International Patent Application WO 98/32859 discloses methods of inducing angiogenesis comprising multiple applications of a pharmaceutical composition comprising (a) a pharmaceutically acceptable carrier and (b) an adenoviral vector comprising a DNA encoding an angiogenic peptide, such that the level of perfusion of blood to the target tissue is enhanced.

There remains a need, however, for improved methods of inducing angiogenesis in a target tissue. The present invention provides such a method. These and other advantages of the present invention, as well as additional inventive features, will be apparent from the description of the invention provided herein.

It is estimated that in 1992 (US), 35.2 million wounds required major therapeutic intervention (Medical Data International, Inc. 1993). Surgical incisional wounds are performed with aseptic technique, and are closed by primary intention. Most repair and heal uneventfully. Many traumatic wounds and cancer extirpations, however, must be left open to heal by secondary intention. Furthermore, chronic wounds have significant tissue necrosis and fail to heal by secondary intention. It is estimated that 5.5 million people in the US have chronic, nonhealing wounds and that their prevalence is increasing secondary to the increase in age-related diseases, the increase in Acquired-immune Deficiency Syndrome (AIDS), and the increase of radiation wounds secondary to cancer intervention. In the US approximately 1.5-2.5 million people have venous leg ulcers; 300,000-500,000 diabetic ulcers; and 2.5-3.5 million pressure ulcers (Callam, M. J. et al. Br. Med. J. 294: 1389-1391 (1987); Phillips, T. J. and Dover J. S., J. Am. Acad. Dermatol. 25: 965-987 (1991); Lees, T. A. and Lambert, D., Br. J. Surg. 79:- 1032-1034 (1992); Lindholm, C. et al., Acta Derm. Venereol (Stockh) 72: 227-230 (1992)). These acute and chronic open wounds require long-term care and procedures that include skin grafting and tissue flaps, debridement, frequent dressing changes and administration of pain medications. This care is costly and labor intensive. Furthermore, these wounds have a severe impact on the patients' quality of life. The chronic dermal ulcerations can cost as much as $40,000 each to heal and more disappointing is that 50% reappear within 18 months of healing. Chronic dermal ulcers are also associated with mortality. As many as 21% of patients in intermediate-care facilities with pressure ulcers die (Bergstrom, N. et al., U.S. Dept. Health and Human Services, Clinical Practice Guideline, vol. 15 (1994)).

Many attempts have been made to produce a composition which can be used to facilitate wound repair. Many of these compositions involve collagen as a component. U.S. Pat. Nos. 4,950,483 and 5,024,841 each discuss the usefulness of collagen implants as wound healing matrices. U.S. Pat. No. 4,453,939 discusses a wound healing composition of collagen with a fibrinogen component and a thrombin component, and optionally fibronectin. U.S. Pat. No. 4,970,298 discusses the usefulness of a biodegradable collagen matrix (of collagen, hyaluronic acid, and fibronectin) for wound healing.

Various other compositions have also been explored for their wound healing capabilities. Kratz et al. (Scandinavian J. of Plastic and Reconstructive Surgery and Hand Surgery 31 (2): 119-123(1997)) used a gel of heparin ionically linked to chitosan. Bartold and Raben (J. Periodontal Res. 31 (3): 205-216 (1996)) studied platelet-derived growth factor (PDGF). U.S. Pat. No. 5,641,483 discloses topical, gel and cream formulations containing human plasma fibronectin for healing of cutaneous wounds. Schultz et al. (Acta Ophthalmologica 70(S202): 60-66 (1992)) disclose a composition of epidermal growth factor (EGF), fibronectin, a synthetic collagenase inhibitor, and Aprotinin.

Various studies involving fibronectin (FN) and/or particular fibronectin peptides and wound healing have also been reported. Many of these studies involve the RGD sequence, part of the cell binding domain of FN (see Schor et al., J. Cell Science 109: 2581-2590 (1996); Steed et al., Diabetes Care 18(1): 39-46 (1995); Spongel et al., Am J. Physiology 267(2): F257-264 (1994); Kartha and Toback, J., Clinical Invest. 90(1): 288-292 (1992); Kishida et al., Biomaterials 13(13): 924-930 (1992)). Other portions of FN have also been studied for wound-healing activity. U.S. Pat. No. 5,198,423 studied the effects of a polypeptide containing a cell binding domain and a heparin binding domain of FN on wound healing. U.S. Pat. No. 4,589,881 studied the effects of a 108 aa polypeptide fragment of FN on wound healing, as well as a biologically active fragment thereof. Sponsel et al. (1994) studied the effect of the tetrapeptide REDV and the peptide LDVPS on wound healing.

However, none of these compounds has proven completely satisfactory because of problems with half-life and adequate delivery of the compounds. The severity of the problem of chronic, nonhealing wounds dictates that continual efforts be made to define new and more effective matrices and methods for facilitating wound healing.

Several, matrices and implant devices have been disclosed to alleviate these problems. U.S. Pat. No. 5,100,668 discusses a controlled release device to regulate the release rate of a growth factor. U.S. Pat. Nos. 6,497,729 and 6,342,051 also use implantable devices to modulate angiogenesis. However, a more efficient method of release of growth factor is desirable at the relevant sites.

VEGF and other angiogenic compositions have been proposed as being useful for individuals suffering from heart ailments such as myocardial infarcts. VEGF has a number of different isoforms. While much attention has focused on the use of proteins such as VEGF, as mentioned above, the delivery of these proteins has proven problematic. Accordingly, it would be desirable to have improved methods of delivery of such proteins that can improve at least one area such as bioavailability, precision of targeting and/or precision of release. Particularly where such improved method of delivering are relatively simple and efficient to obtain.

### SUMMARY OF THE INVENTION

The present invention is directed to using a device that will efficiently bind a protein under acidic conditions, while permitting its release under normal physiological conditions. One can use any protein that contains a domain that binds to heparin or fibronectin, preferably heparin, where that binding is pH dependent such as occurs with VEGF. Preferably, the protein contains or is altered to contain a heparin-binding consensus sequence such as XBBBXXBX or XBBXBX, where B is a basic amino acid residue such as Lys, Arg, or His, and X is any amino acid residue. Preferably, the protein contains or is altered to contain a pH sensitive residue. For example, His has a pH sensitive ionizable moiety. The fibroblast growth factor family of heparin-binding proteins have a stretch of about nine amino acids that are believed to play a role in heparin-binding specificity. [Ashikari-Hada, S., et al., J. Biol. Chem (2004); Luo, Y. et al., Biochemistry 37:16506-15(1998)]. These regions typically include two Gly residues and 2-5 additional basic amino acid residues (e.g. Lys or Arg). The glycine-like box ranges from six to twenty amino acids, preferably seven to twelve, more preferably seven to ten amino acid residues. VEGF₁₂₁ and VEGF₁₆₅ show pH dependent heparin binding and have a “glycine-like box” (residues 84-92) (SEQ ID NO:1 Lys-Pro-His-Gln-Gly-Gln-His-Ile-Gly) containing two pH sensitive ionizable His residues in place of formally basic residues. Thus, for example, a matrix containing heparin or heparin-related compound under an acidic internal environment allows efficient binding of VEGF, which is then released in active form at physiological pH. One can use this device at desired sites, namely those at or near physiologic pH to release the VEGF, and thereby to promote angiogenesis. The device is made at a low pH, preferably from pH 4.5 to pH 6.0, more preferably about pH 5.0 to pH 6.0, and still more preferably at pH. 5.5.

### BRIEF DESCRIPTION OF THE DRAWINGS

FIGS. 1A-D illustrate ¹²⁵I-VEGF₁₆₅ and ¹²⁵I-VEGF₁₂₁ binding to BAEC at various pHs. Bovine aortic endothelial cells (BAEC) were incubated at the pH indicated at 4° C. for 2.5 h. Binding assays were performed with ¹²⁵I-VEGF₁₆₅ (FIG. 1A and C) or ¹²⁵I-VEGF₁₂₁ (FIGS. 1B and D). VEGF interactions involving HSPG were determined with a high salt, neutral pH wash (FIGS. 1A and B). Following the high salt wash, the remainder of VEGF bound to cell surface receptors was accounted for by extracting cells in 1 N NaOH (FIGS. 1C and D). Samples were quantitated in a γ counter. Cells maintained in exact conditions as experiments without the addition of radiolabled VEGF were trypsinized and counted in a Coulter Counter to normalize samples to cell number. Representative data are presented as the mean of triplicate determinations±standard error of mean.

FIGS. 2A and B show ¹²⁵I-VEGF₁₆₅ and ¹²⁵I-VEGF₁₂₁ binding to chinese hamster ovary (CHO) cells at various pHs. Binding assays were conducted on confluent CHO-K1 cells. Cells were incubated at various pHs as indicated at 4° C. for 2.5 h. Binding assays were performed with ¹²⁵I-VEGF₁₆₅ (FIG. 2A) or ¹²⁵ I-VEGF₁₂₁ (FIG. 2B). VEGF interactions involving HSPG were determined with a high salt, neutral pH wash. Samples were quantitated in a γ counter. Samples were normalized to cell number. Representative data are presented as the mean of triplicate determinations±standard error of mean.

FIGS. 3A and B show ¹²⁵I-VEGF₁₆₅ and ¹²⁵I-VEGF₁₂₁ binding to BAEC deposited ECM at various pHs. BAEC were grown to confluence for 3 days in culture. Cells were extracted leaving behind the deposited ECM. Binding assays were conducted on ECM. Matrices were incubated at various pHs as indicated at 4° C. for 2.5 h. Binding assays were performed with ¹²⁵I-VEGF₁₆₅ (FIG. 3A) or ¹²⁵I-VEGF₁₂₁ (FIG. 3B). VEGF interactions involving HSPG were determined with a high salt, neutral pH wash. Samples were quantitated in a γ counter. Data are presented as the mean of triplicate deteriorations±standard error of mean.

FIGS. 4A-C show VEGF₁₆₅, VEGF₁₂₁ and EGF elution through heparin-Sepharose column at pH 7.5 and pH 5.5. Heparin-Sepharose columns were equilibrated in pH 7.5 or pH 5.5 buffers. ¹²⁵I-VEGF₁₆₅ (FIG. 4A), ¹²⁵I-VEGF₁₂₁ (FIG. 4B) or ¹²⁵I-EGF (FIG. 4C) were incubated in either pH 7.5 or pH 5.5 buffers for 20 min and then applied to the columns. Columns were washed with the incubation buffer and collected as Flow Through (black bars). Columns were then washed with either pH 7.5 or pH 5.5 buffer and collected (white bars). The remaining ¹²⁵I-VEGF bound to the columns was quantitated (grey bars). EGF was only washed with the incubation buffer (white bars). Data from one experiment is presented as a representative of similar results from 3 separate experiments.

FIGS. 5A-D show the effects of heparin on VEGF₁₆₅ and VEGF₁₂₁ binding to BAEC at pH 7.5, 6.5 and 5.5. Confluent BAEC were incubated at pH 7.5 (⋄), pH 6.5 (□), or pH 5.5 (Δ) in the presence of various concentrations of heparin and 0.16 nM ¹²⁵-VEGF₁₆₅ (FIGS. 5A and B) or 0.14 nM ¹²⁵I-VEGF₁₂₁ (FIGS. 5C and D) for 2.5 h at 4° C. VEGF interactions involving HSPG were determined with a high salt, neutral pH wash. Samples were quantitated in a γ counter. Data was normalized to cell number. The data presented in panels 5B and 5D represent the percent of VEGF binding compared to the binding in the absence of heparin (Percent Bound=(VEGF bound with heparin/VEGF bound without heparin)×100). Representative data are presented as the mean of triplicate±standard error of the mean.

FIGS. 6A and B show Erk½ phosphorylation in response to VEGF₁₆₅ and VEGF₁₂₁ at pH 7.5, 6.5 and 5.5. Effect of pH on VEGF stimulated Erk½ activation. Subconfluent BAEC were treated with pH 7.5, pH 6.5 or pH 5.5 buffers for 90 min at 37° C. prior to the addition of 0.6 nM VEGF₁₆₅ (FIG. 6A) or 0.7 nM VEGF₁₂₁ (FIG. 6B) and incubated at 37° C. for the various times indicated. Cells were extracted and SDS-PAGE (12%) was conducted and transferred to Immobilon membranes. Blots were hybridized with anti-phospho-Erk½ antibody and visualized with ECL reagent. Blots were stripped of antibodies and rehybridized with anti-Erk½ antibody and visualized with ECL reagent.

FIGS. 7A and B show recovery of Erk½ phosphorylation in response to VEGF₁₆₅ incubated at pH 5.5. Effect of pH on VEGF₁₆₅ activation of Erk½ (FIG. 7A). Subconfluent BAEC were treated with pH 7.5 or 5.5 buffers for 90 min at 37° C. VEGF₁₆₅ (23.8 nM) was incubated at pH 5.5 for 20 min prior to the addition to cells at a final concentration of 0.6 nM. Cells were incubated at 37° C. for the various times indicated. Effect of pH on BAEC activity (FIG. 7B). Subconfluent BAEC were

incubated at pH 5.5 or pH 7.5 for 30 min followed by 60 min incubation at pH 5.5 or pH 7.5. VEGF₁₆₅ (0.6 nM) was added to cells for 2, 5 or 10 min. Cells were extracted and subjected to SDS-PAGE (12%) and transferred to Immobilon membranes. Blots were hybridized with anti-phospho-Erk½ antibody and visualized with ECL reagent. Blots were stripped of antibodies and rehybridized with anti-Erk½ antibody and visualized with ECL reagent.

FIG. 8 show the effect of heparinase treatments on ¹²⁵I-VEGF₁₆₅ and ¹²⁵I-VEGF₁₂₁ binding to BAEC at pH 5.5. BAEC were treated with 0.5 units/mL of heparinase II or III; or 1 μg/mL of heparinase I or various combinations of each for 1 h at 37° C. prior to conducting binding assays with ¹²⁵I-VEGF₁₆₅ (0.16 nM) and ¹²⁵I-VEGF₁₂₁ (0.14 nM) at pH 5.5. After ¹²⁵I-VEGF₁₆₅ (black bars) and ¹²⁵I-VEGF₁₂₁ (white bars) binding occurred, the cells were washed with a high salt, neutral pH buffer to release bound VEGF. Samples were quantitated in a γ counter. Treatments are labeled as: NT=No Treatment, I=heparinase I, II=heparinase II, III=heparinase III. Representative data are presented as the mean of triplicate determinations±standard error of the mean, and % Bound was defined as: (VEGF bound in heparinase treated/VEGF bound in untreated)×100. A one way analysis of variance (ANOVA) was conducted and revealed that the enzyme treatments caused a statistically significant reduction in VEGF₁₆₅ and VEGF₁₂₁ binding (p<0.01). However, ANOVA of the various enzyme treatments (I, II, III, or combinations) revealed no significant differences between treatments (p=NS).

FIG. 9 shows the effect of pH on ¹²⁵I-VEGF₁₆₅ and ¹²⁵I-VEGF₁₂₁ binding to fibronectin. Fibronectin coated dishes were prepared. Binding assays were conducted using 0.12 nM ¹²⁵I-VEGF₁₆₅ (black bars) and 0.14 nM ¹²⁵I-VEGF₁₂₁ (white bars). Total binding was determined by extraction in 1 N NaOH. Samples were quantitated using a γ counter. Representative data are presented as the mean of triplicate±standard error of the mean. ANOVA followed by the Newman-Keul's multiple comparison t-test was run revealing that VEGF₁₆₅ and VEGF₁₂₁ binding was significantly different at the three pHs tested (ANOVA, p<0.001 for both VEGF₁₆₅ and VEGF₁₂₁; Newman-Keul's, p<0.001 for all comparisons).

FIGS. 10A and B show heparinase III treatment of fibronectin. Fibronectin-coated dishes were treated with 0.5 units/mL of heparinase III. Binding assays were conducted using 0.12 nM ¹²⁵I-VEGF₁₆₅ (FIG. 10A) and 0.14 nM ¹²⁵I-VEGF₁₂₁ (FIG. 10B). Total binding was determined by extraction in 1 N NaOH. Samples were quantitated using a γ counter. Representative data are presented as the mean of triplicate±standard error of the mean. Heparinase III treatment showed no statistically significant effects on VEGF₁₆₅ or VEGF₁₂₁ binding (based on paired student's t-test).

FIGS. 11A and B show VEGF₁₆₅ and VEGF₁₂₁ binding to fibronectin, collagen, and BSA at neutral and acidic pH. Binding assays were conducted on fibronectin (FN), collagen type I (Col), and bovine serum albumin (BSA). ¹²⁵I-VEGF₁₆₅ (0.12 nM) (FIG. 11A) and ¹²⁵I-VEGF₁₂₁ (0.14 nM) (FIG. 11B) were added to dishes for 2.5 h at 4° C. at pH 7.5 and pH 5.5. Total binding was determined by extraction in 1 N NaOH. Samples were quantitated using a γ counter. Representative data are presented as the mean of triplicate±standard error of the mean. Binding of VEGF₁₆₅ and VEGF₁₂₁ were significantly different at the two pHs tested (p<0.01), while there was no significant effects of pH on binding to collagen or BSA (based on paired student's t-test).

FIGS. 12A and B show effects of heparin on VEGF binding to fibronectin and collagen. Binding assays were conducted on fibronectin. ¹²⁵I-VEGF₁₆₅ (0.12 nM) was added to fibronectin (♦) and collagen (▴); and ¹²⁵I -VEGF₁₂₁ (0.14 nM) was added to fibronectin (□) and collagen, (◯) for 2.5 h at 4° C. in the presence of various concentrations of heparin at pH 7.5 (FIG. 12A) and pH 5.5 (FIG. 12B). Total binding was determined by extraction in 1 N NaOH. Samples were quantitated using a γ counter. Representative data are presented as the mean of triplicate±standard error of the mean.

FIGS. 13A-D show VEGF dissociation from fibronectin in the presence or absence of heparin with increasing pH. Binding assays were conducted on fibronectin at pH 5.5 with ¹²⁵I-VEGF₁₆₅ (0.6 nM) in the absence of heparin (FIG. 13A) and in the presence of heparin (FIG. 13B); and ¹²⁵I-VEGF₁₂₁ (0.7 nM) in the absence of heparin (FIG. 13C), and in the presence of heparin (FIG. 13D). After binding, fresh buffer was added to fibronectin at pH 7.5 (♦), pH 6.5 (□), and pH 5.5 (⋄) without heparin and collected at various time points. Samples were quantitated using a γ counter. Representative data are presented as the mean of triplicate±standard error of the mean.

FIGS. 14A and B show VEGF activity after binding to fibronectin. Binding assays were-conducted on fibronectin-coated dishes with 3.0 nM, of VEGF₁₆₅ (FIG. 14A) and 3.5 nM of VEGF₁₂₁ (FIG. 14B) in the presence of heparin 1 μg/mL) at 37° C. for 1 h. Meanwhile, subconfluent BAEC were treated with pH 7.5 or pH 5.5 buffers for 90 min at 37° C. After binding occurred on fibronectin, unbound VEGF was removed and fresh buffer was added to the fibronectin-coated dishes and the incubation continued for 20 min. Media containing released VEGF was collected and added to BAEC for 10 min. Control experiments were conducted on quiescent BAEC by adding 0.6 nM VEGF₁₆₅ (FIG. 14A) or 0.4 nM VEGF₁₂₁ to cells for 10 min at pH 7.5 or pH 5.5. Cells were extracted and SDS-PAGE (12%) was conducted and transferred to Immobilon membranes. Blots were probed with anti-phospho-Erk½ antibody and visualized with ECL reagent. Blots were stripped of antibodies and reprobed with anti-Erk½ antibody.

FIGS. 15A and B show VEGF₁₆₅ binding to BAEC at pH 5.5 followed by activation at various pHs. VEGF₁₆₅ (0.6 nM) was added to cells at pH 5.5 for 60 min at 37° C. Unbound VEGF₁₆₅ was removed. Cells were incubated for 10 min at pH 7.5, 7.0, 6.5, 6.0, or 5.5 (FIG. 15A). FIG. 15B represents the stimulation of subconfluent BAEC by directly adding 0.6 nM VEGF₁₆₅ (no prior binding occurred) to the cells at the indicated pH (7.5-5.5). Cells were extracted and subjected to SDS-PAGE (12%) and transferred to Immobilon membranes. Blots were-probed with anti-phospho-Erk½ antibody and visualized with ECL reagent. Blots were stripped of anitibodies and reprobed with anti-Erk½ antibody.

FIGS. 16A and B show a model of pH effects on VEGF interactions. FIG. 16A is a schematic representation of VEGF [symbol] interactions with fibronectin [symbol] and HSPG [symbol] at acidic and neutral pH in relation to its ability to stimulate VEGF receptors on endothelial cells. At low pH, there are high levels of VEGF bound to fibronectin and HSPG in the ECM with little receptor activation as indicated by Erk½ activation. The switch to high pH (6.5-7.5) is accompanied by the release of VEGF from the ECM and stimulation of VEGF receptors leading to Erk½ activation. FIG. 16B is a schematic representation of how pH sensitive binding of VEGF contributes to directed angiogenesis into a hypoxic tissue. VEGF is distributed such that the area of the tissue that is most hypoxic (lowest pH) would contain the most VEGF because of the high binding to fibronectin and HSPG as well as the increased expression of VEGF. Thus, variable matrix binding of VEGF would allow a stable VEGF gradient to be established from the most acidic to the least acidic region of the tissue. Hence, the region of the tissue that is closest to the existing vasculature would contain the least amount of deposited VEGF, yet this VEGF would be in the active state such that it could participate in initiating angiogenesis. As the blood vessel grows into the acidic tissue the extracellular pH would increase causing the release of the high levels of stored VEGF, further propagating blood vessel growth.

FIG. 17 shows cumulative percent release of VEGF₁₆₅ from a device of the present invention after 1 day at varying pH.

FIG. 18 shows cumulative percent release of VEGF₁₂₁ from a device of the present invention after 3 days at varying pH.

FIG. 19 shows cumulative percent release of VEGF₁₆₅ from a device of the present invention after 6 days at varying pH.

### DETAILED DESCRIPTION OF THE INVENTION

We have now discovered that an extracellular matrix (ECM) that comprises heparin and heparin-related compounds such as heparan sulfate (HS) as well as oligosaccharides derived from heparin and heparan sulfate can bind certain proteins under an acidic environment and release them at or near physiologic pH. The proteins are proteins containing a heparin-binding domain that is pH dependent such as VEGF. Preferably, VEGF₁₆₅ and VEGF₁₂₁. The matrix may be used to deliver a protein at a specific site where it will be released at physiologic pH.

For example, a preferred type of protein is an angiogenic protein (sometimes referred to as a factor that promotes angiogenesis). In a preferred embodiment, the factor that promotes angiogenesis is VEGF. Preferably the acidic internal environment is between about pH 4.0 and about pH 6.5, more preferably between about pH 5.0 and pH 6.0. In a more preferred embodiment, the internal pH is about 5.5. In one embodiment of the present invention, the ECM also contains factors that help stabilize the angiogenic protein. In one preferred embodiment, such factors comprise heparin and fibronectin. The ECM of the invention is herein termed a “device”.

The extracellular matrix of the present invention is composed of the formulation of gels or devices for topical application to exposed wounds or implantation in diseased tissue. Gels may be comprised of heparin and fibronectin alone or within a number of commercially available materials, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates, gelatin, and cellophane (cellulose with a plasticizer). Additionally, the angiogenesis enhancing agents may be incorporated into polymeric microspheres or devices for implantation made of biocompatible degradable or non-degradable polymers, i.e. polyglycolides, polyanhydrides, polyethylene vinyl acetate, and co-polymers of lactides/glycolides in various ratios.

Heparin is a heterogeneous group of straight-chain anionic mucopolysaccharides, called glycosaminoglycans. In a most preferred embodiment, heparin is incorporated within the device. Heparin is a highly sulfated linear polysaccharide. The protein core has multiple glycosamnmoglycan (GAG) chains attached to serine residues. The average GAG chain has a molecular weight that average 15,000 daltons. Commercial heparin comprises polymers of repeating disaccharide units; D-glucosamine-L-iduronic acid and D-glucosamine-D-glucuronic acid. Heparin is strongly acidic because it has a high content of covalently linked sulfate and carboxylic acid groups.

Related to heparin, and therefore also useful with this invention are substances such as heparan sulfate, which is referred to as a heparin-related compounds. As used in the specification, unless specifically distinguished, the term heparin is intended to apply to both heparin and heparin-related compounds such as heparan sulfate, heparan sulfate proteoglycans, as well as other oligosaccharides derived from heparin and heparan sulfate. The heparin-related compound is preferably a heparin-related oligosaccharide. Preferably the length is 8-16 sugars.

In another embodiment, one uses oligosaccharides that have been chemically modified so that the specific sulfation pattern has been altered (i.e. heparin and oligosaccharides where sulfate is chemically removed from the N-position of the glucosamine residues, or from the 2-0 position of the iduronic/glucoronic acid residue, or from the 6-0 position of the glucosamine, or from the 3-0 position of the glucosamine.

In one embodiment the heparin-related oligosaccharides are devoid of anticoagulant activity. This anticoagulant activity can be defined by the absence of antithrombin III binding activity.

Heparin and heparin-related compounds interact with and stabilize many proteins. For example, heparin binds to both fibroblast growth factor (FGF) and under certain pH's to VEGF. Furthermore, the stabilizing effect of heparin related substances, combined with its ability to bind proteins with heparin-binding domains such as FGF and VEGF allows the formation of a stable bound protein such as a growth factor carrier in a solid form.

More specifically, heparin can bind to the surface of a wide variety of materials by chemical and enzymatic linking. Thus, growth factors that can bind to heparin, such as FGF and VEGF can now be incorporated into the device through interactions with heparin.

There are numerous examples of backbone matrices suitable for use in the subject invention. These examples include fibrin, hyaluronic acid, polyethylene glycolpoly-L-glycol, and poly-L-lactate. Hyaluronic acid is commercially available as a dry (for example, lyophilized) powder, and can be reconstituted to a hyaluronic acid gel (in accordance with manufacturer's suggestions) for use in the subject invention. Depending upon the viscosity desired, a hyaluronic acid gel having about 5 milligrams to about 50 milligrams of hyaluronic acid per milliliter of reconstituting solution can be used. At 5 milligrams/milliliter, the hyaluronic acid gel will be more liquid, and at 50 milligrams/milliliter the hyaluronic acid gel will become more viscous and less easy to manipulate. The use of the gel will, in part, dictate the desired viscosity. If the extracellular matrix can be “poured” into and contained in a wound area, then a more liquid form of the hyaluronic acid gel will be satisfactory. If the extracellulat matrix is “spread” over and/or into a wound area, then a more viscous form of the hyaluronic acid gel will be desirable. In either case, a dressing of some form will often cover the applied extracellular matrix to help prevent contamination and infection of the wound. It should be readily apparent that the extracellular matrix itself (and each of its components) must be sterile (free of biological and/or chemical contamination) to also prevent contamination and infection of the wound.

Preferably, the hyaluronic acid gel is provided as a gel having about 20 milligrams of dry hyaluronic acid per milliliter of reconstituting solution. Suitable reconstituting solutions include, for example, sterile distilled water, sterile phosphate buffered saline (PBS), or a cell culture medium.

As used herein, “hyaluronic acid” is intended to include the various forms of hyaluronic acid (HA) known in the art. These various forms include HA chemically modified (such as by cross-linking) to vary its resorbtion capacity and/or its ability to be degraded. Optimal HA formulations will be resorbable in a few days to a week.

Other components may also be incorporated, such as poly(ethylene oxide) (PEG), to minimize protein adsorption. Poly(ethylene oxide) is most readily incorporated into the hydrogel, for example, by co-polymerization of a vinyl monomer having poly(ethylene oxide) side chains, for example poly(ethylene glycol) methacrylate (which is commercially available from Aldrich Chemical Co.), or a divinyl-terminated poly(ethylene glycol) macromonomer. Copolymerization of HEMA and poly(ethylene glycol) methacrylate in the presence of AIBN yields a more flexible, unhydrated copolymer. The optimal molecular weight and content of poly(ethylene oxide) for each application can be determined by protein adsorption studies.

To provide further chemical functionality on the bioactive polymer layer, particularly a hydrogel layer, either polyvinyl alcohol or polyethylene imine may be employed as macromolecular surfactants. Where hydroxyl functionalities are available, the coupling is promoted by tresylation. Poly(ethylene oxide) may also be grafted to hydroxyl groups on the surface of the polymer layer by tresylation coupling with Jeffamine, an amine-terminated poly(ethylene oxide) commercially available from Huntsman.

In one embodiment of the present invention, fibronectin is incorporated into the ECM. Fibronectin is an ECM protein dimer composed of two nearly identical 250 kDa subunits. Fibronectin has been shown to play a role in a number of cellular functions, including in cell adhesion, migration, growth and differentiation. Particular to the present invention, fibronectin has been suggested to play an important role in angiogenesis through its ability to recognize endothelial cells. (Tonnesen, M. G. et al., J. Investig. Dermatol. Symp. 5: 40-46, 2000). Fibronectin contains a heparin binding domain and has been shown to interact with both heparin and VEGF. One can use a fibronectin fragment that binds VEGF.

Any protein that binds to heparin may be used in the present invention. Preferably, the protein binds heparin in a pH dependent manner. For example, proteins that possess a heparin-binding domain or are engineered to contain such domains may be used in the present device. A heparin-binding domain preferably is based on a consensus sequence such as XBBBXXBX or XBBXBX (where B=a basic amino acid residue such as lysine, arginine, or histidine) (Hileman et al., Bioessays, 20: 156-67, 1998, Cardin et al., Methods Enzymol., 203: 556-83, 1991, and Cardin et al., Atheriosclerosis, 9:21-32, 1989). Certain proteins containing such domains show or can be engineered to show pH dependence as a result of for example ionizable histidine residues within, or in close proximity to, the heparin-binding domain. For example, in VEGF, the sequences: XBXBXXXH or HXBXXXBXBBXBX (where H=His) could confer pH dependent heparin-binding. The fibroblast growth factor family of heparin-binding proteins have a stretch of about nine amino acid residues falling within a heparin-binding consensus pattern. These regions typically include two Gly residues and 2-5 additional basic amino acid residues (e.g., Lys or Arg), referred to as a Glycine box. VEGF₁₂₁ and VEGF₁₆₅ show pH dependent heparin binding. These VEGF isomers have a similar “glycine-like box” (residues 84-92, SEQ ID NO:1) that contain two pH sensitive ionizable His residues in place of formally basic residues. Although not wishing to be bound by theory, the His residues in VEGF may result in conformational changes at different pH's that expose or mask the heparin-binding domain. One can use proteins that naturally exhibit pH dependent heparin binding or modify the protein, e.g., by adding or substituting His residues to result in a pH dependent binding such as VEGF.

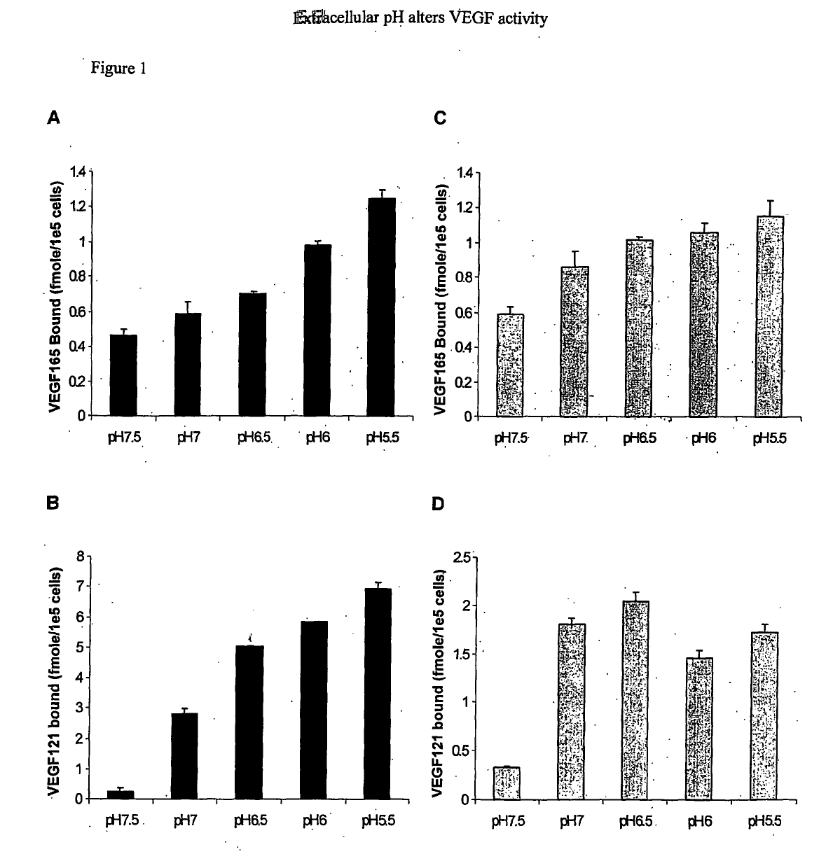

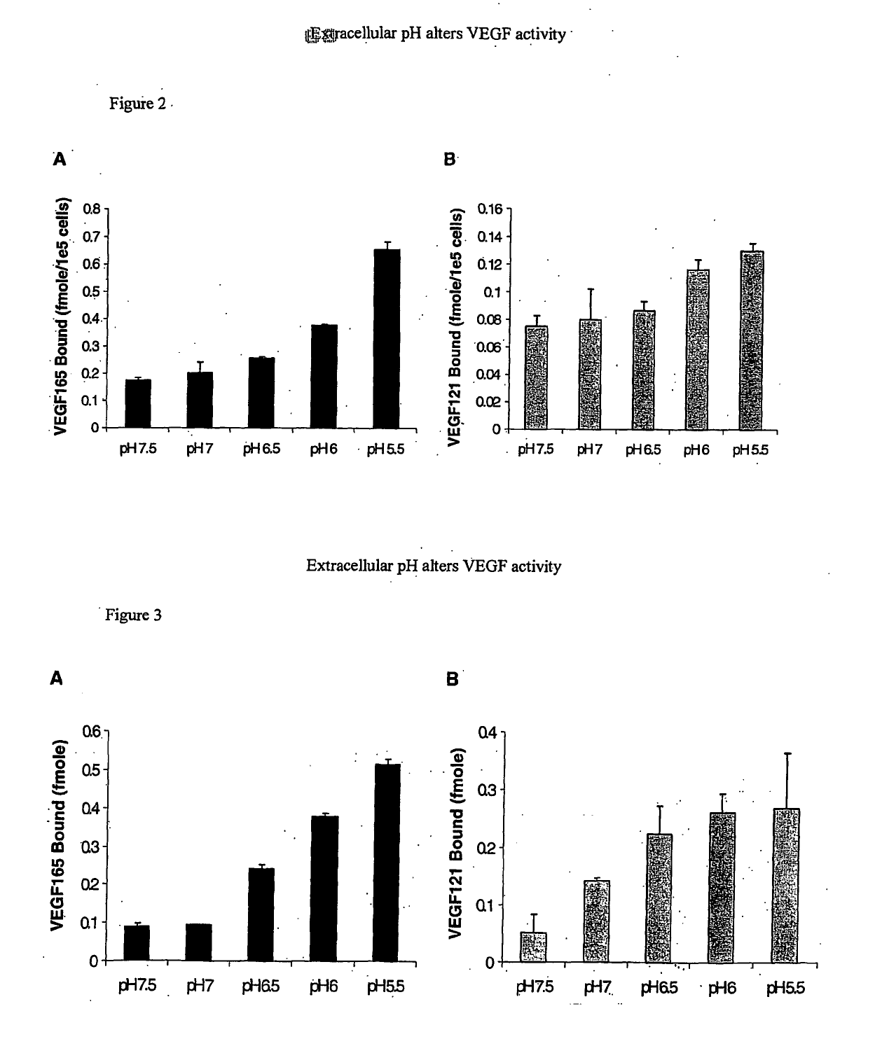

VEGF is a dimeric glyprotein having several isoforis ranging from 121 to 206 amino acids (VEGF₁₂₁, VEGF₁₄₅, VEGF₁₆₅, VEGF₁₈₃, VEGF₁₈₉, and VEGF₂₀₆ [Robinson, C. and Stringer, S., Journal of Cell Science, 114 (5) (2001)]. The isoforms can vary in their physiochemical properties such as binding to heparin and heparan sulfate proteoglycans (HSPG). Both VEGF₁₂₁ and VEGF₁₆₅ show little binding at pH 7.5 but as the pH decreases, there is an increase in binding to heparin and HSPG (see FIGS. 1 and 3). VEGF₁₂₁ transport through an extracellular matrix is relatively unimpeded when compared to VEGF₁₆₅. Although not wanting to be bound by theory, this is probably due to interactions with heparin-related compounds such as HSPG. The VEGF binding at acidic pH is not dependent on VEGF receptors.

The VEGF isoforms are potent angiogenic factors stimulating endothelial proliferation, migration survival and permeability.

We also found that in the extracellular matrix there are other factors in addition to heparin and heparin-related compounds that affect binding of a protein such as VEGF. For example, fibronectin. VEGF isomers ₁₆₅ and 121 show pH dependent binding to fibronectin which is substantially increased at a pH of 5.5 when compared to pH 7.5 (approximately 8 fold). Again, there are differences between the different isomers. The addition of heparin increases VEGF binding to fibronectin at pH 5.5 for both VEGF₁₂₁ and ₁₆₅. However, when heparin is added at pH 7.5, it does not increase VEGF₁₂₁ binding to fibronectin but does increase VEGF₁₆₅ binding to fibronectin. The isomers disassociate from both fibronectin and heparin as pH increases. Once released, and at physiological pH, VEGF activity is present. For example, we have demonstrated that VEGF can be stored in a device according to the invention, it can be released and it will still stimulate Erk½ phosphorylation. This shows that the device of the present invention can be used to bind a desired protein and release it at the appropriate time and pH.

For example, hypoxic tissue has a low pH. By adding one of the present devices containing an angiogenic protein such VEGF to such a site, the release of VEGF will stimulate blood vessel growth which will raise the pH which will result in the enhanced release of more VEGF resulting in greater blood vessel growth which in turn raises the pH, which in turn releases more VEGF which in turn results in greater blood vessel growth at the desired site. Thus, proteins that normally contain pH dependent heparin-binding domains or engineered proteins where these domains are added can be effectively stored and delivered to sites in need of angiogenesis using the pH regulated drug delivery device of the present invention. In an alternative embodiment, one can use the device to bind to a protein at a low pH and thus take the protein out of circulation.

The extracellular matrix may comprise other components which further enhance the angiogenic/cell migration effect of the device. Such additional components include, for example, platelet derived growth factor. Suitable examples of growth factors include Fibroblast Growth Factor (FGF) (more particularly, acidic Fibroblast Growth Factor (aFGF) and basic Fibroblast Growth Factor (bFGF)), Transforming Growth Factor (TGF) (particularly, Transforming Growth Factor-Beta)(TGFβ), Placental Growth Factor, Platelet-derived Endothelial Cell Growth Factor, and Vascular Endothelial Growth Factor (VEGF). Other angiogenic mediators include Angiogenin, and cytokines such as Tumor Necrosis Factor-alpha and Interleukin-8 (IL-8). The angiogenic mediator is not limited to peptides such as cytokines or growth factors, but includes other substances such as modified peptides (e.g., glycoproteins or lipoproteins), ribozymes, polynucleotides, and other substances that induce angiogenesis.

The present invention also provides a method to stimulate angiogenesis at a clinically relevant site using the device of the present invention. Contacting a site in need of angiogenesis with the above described device will result in stimulating angiogenesis at sites in need thereof such as, for example, a wound, the heart following myocardial infarct, the lower extremities of individuals with peripheral vascular disease, or in individuals following stroke or with necrotic tissue.

The invention further provides a method of promoting angio genesis at a desired site. The matrix bound angiogenic protein, VEGF, binds to ECM proteins (e.g. heparin and fibronectin) with increasing affinity at acidic pH. Acidic pH may range from about 4.0 to about 6.5. Furthermore, VEGF is released at about physiological pH, e.g., above pH 6.5, preferably about 7.0 to about 7.5. Such release does not alter the biological activity of the angiogenic protein. For example, VEGF binds to both heparin and fibronectin at acidic pH. Upon exposure to more physiological pH, the bound VEGF is released from both fibronectin and heparin. The released VEGF retains its ability to stimulate extracellular-regulated kinases ½ (Erk½) phosphorylation in endotheblial cells. Thus, the device of the present invention may be used to deliver angiogenic proteins and growth factors to sites in need of vascularization.

The device formulated at an acidic pH, serves to store angiogenesis promoting factors at low pH. Upon exposure to a more physiologic pH, the angiogenic factors are released and are able to promote angiogenesis. The placement of the device at a site in need of angiogenesis generates a gradient of VEGF, whereby exposure to a more physiological pH releases active VEGF, thus promoting angiogenesis near the device. As new vessels move into regions of low pH (and high levels of stored VEGF), the corresponding extracellular pH rises, resulting in the conversion of the stored VEGF to the active form, further stimulating the directed growth and migration of the new vessel. Therefore, a dynamic system of reversible VEGF storage and activation within the ECM can contribute to the positional growth of new blood vessels to undercirculated/hypoxic/acidic regions of tissue via the pH sensitive binding of a bound protein such as VEGF in the device of the invention.

Thus, the methods of the present invention allow for the controlled delivery of angiogenic promoting factors to places in need of new vasculature.

There are a many instances when angiogenesis is desired. For example, in patients suffering from heart disease such as myocardial infarcts, heart failure, diseased myocardial tissue, etc. the increased vascularization has proven to be beneficial. In another embodiment, enhancing wound healing, which can comprise applying the extracellular matrix (as described herein) near a wound, is useful. As discussed above, the method of applying the extracellular matrix to the wound may vary depending on the type and location of the wound as well as the viscosity of the extracellular matrix. Preferably, the extracellular matrix is viscous enough to be “spread” over the wound and will not run off after application.

The matrix may be used to increase vascularization in patients in need thereof. Thus, the methods of the invention are useful for the treatment of diseases or conditions that benefit from increased blood circulation, for providing a vascularized site for transplantation, for enhancing wound healing, for decreasing scar tissue formation, i.e., following injury or surgery, for conditions that may benefit from directed suppression of the immune response at a particular site, and the like.

Any condition that would benefit from increased blood flow are encompassed such as, for example, gangrene, diabetes, poor circulation, arteriosclerosis, atherosclerosis, coronary artery disease, aortic aneurysm, arterial disease of the lower extremities, cerebrovascular disease, etc. In this manner, the methods of the invention may be used to treat peripheral vascular diseases. Likewise, the device is useful to treat a diseased or hypoxic heart, particularly where vessels to the heart are obstructed. Other organs with arterial sclerosis may benefit from an application of the device. Likewise, organs whose function may be enhanced by higher vascularization may be improved by an application of the device. This includes kidneys or other organs which need an improvement in function. In the same manner, other targets for arterial sclerosis include ischemic bowel disease, cerebrovascular disease, impotence of a vascular basis, and the like. Additionally, formation of new blood vessels in the heart is critically important in protecting the myocardium from the consequences of coronary obstruction. Application of the device into or near a site of ischemic myocardium can enhance the development of collaterals, accelerate the healing of necrotic tissue and prevent infarct expansion and cardiac dilatation.

One uses a device that contains a sufficient amount of the bound protein to release a pharmaceutically effective amount of the protein such as VEGF. A pharmaceutically effective amount of a protein typically ranges from 0.1 to 100,000 μg per kilogram of bodyweight of the recipient of the device per day, preferably in the range of from 1 to 10,000 μg of protein per kilogram bodyweight of recipient per day.

An effective amount of the device is applied to a site in a mammal where vascularization is desired. An effective amount is an amount necessary to stimulate the flow of blood to the desired anatomic site. The device may be used to improve vascularization at a transplant site so that a blood supply is already available for the transplanted cells, tissues, or organs in the recipient. However, the device of the present invention may be re-applied to the transplant site at the time of the procedure so that neovascularization occurs within a few days, generally about 4 to 7 days. The vascularization effect of the device increases the likelihood of long-term cell and organ viability in a recipient.

The methods of the invention can be used to increase vascularization in any mammal in need thereof. Mammals of interest include humans, dogs, cows, pigs, cats, sheep, horses, etc., particularly humans.

Any means may be used to apply or administer the device to the desired anatomic site. The amount of device applied will vary depending upon the amount of circulation needed (for example, the size of the organ or tissue to be implanted in the recipient, the area of the site, etc.), the weight and size of the recipient, the condition being treated, and the like. An effective amount of the device is an amount that promotes the desired amount of vascularization or blood flow and prevents an immune response and the formation of scar tissue.

The device is suitable for use in the transplantation of cells within a transplant device such as described in U.S. patent application Ser. No. 08/568,694, which is herein incorporated by reference in its entirety. A transplant device is any device designed to contain and protect cells transplanted into a host organism for the production of hormones or other factors. Examples of other transplant devices suitable for use with the device include those described in U.S. Pat. Nos. 5,686,091, 5,676,943 and 5,550,050. However, it is also recognized that the device may be used as the sole transplant vehicle without using such transplantation devices.

The methods of the invention are useful for the stimulation of new blood vessels without the presence of immune cells and the characteristic immune response. Thus, the use of the device of the invention results in vascularization without the formation of scat tissue. Therefore, the device of the invention may be utilized in any physiological setting where the formation of blood vessels is desired.

Cardiac and stroke patients may benefit by an increase in vascularization. Thus, the device may be used to improve circulation in post stroke or heart attack victims.

Because the device is beneficial in preventing or reducing the inflammatory response, it may be used to treat chronic inflammatory diseases, including rheumatoid arthritis, atherosclerosis, tuberculosis, chronic lung diseases, autoimmune diseases, particularly rheumatoid arthritis and lupus erythematosis. For treatment, the device is injected or applied at the site of interest. For example, to reduce arthritis, the device may be injected into a joint in need thereof.

As indicated previously, the device is useful to prepare a transplant site for tissues or organs of interest. Such organ transplants include, but are not limited to, pancreas, kidney, heart, lung, liver, etc. The device may also be used in combination with other implants as a surgical adhesion barrier. This finds particular use with breast implants. Coating the implant in the device prevents or reduces the likelihood of scar tissue formation and adhesion, thus reducing pain and inflammation following surgery. Likewise, the device may serve as an adjunct to provide vascularization to a cellular implant. Such cells in the implant may be native or genetically modified.

Following in vitro fertilization, the embryo is implanted in a female for gestation. The methods of the invention can be used to prepare a vascularized bed for transplantation. In this embodiment, the uterine wall is contacted with the device to promote blood vessel formation prior to implantation of the embryo. The device may be applied prior to, concurrently, or after implantation.

As indicated earlier, the device enables vascularization without stimulating inmune cells. Thus, the device finds use in promoting wound healing. The device provides new blood vessel growth and fibroblasts to the site without the attraction of immune cells. The device prevents inflammation while promoting wound healing. Any tissue, or site, in need of repair or healing may benefit from application of the device to the site. Sites include those resulting from injury or surgery. The device may be applied to internal, or external surgical or injury sites to reduce the pain accompanying a classic inflammatory response, and to reduce scar tissue formation.

The device is also beneficial for superficial wound healing. Thus, it may be useful to apply to skin ulcers, burn areas, ulcers that form secondary to peripheral vascular disease, or other tissue damage.

The device of the present invention may be delivered to tissues in need of angiogenesis in any number of ways. For example, the device may be delivered topically, implanted, injected, inhaled, or swallowed.

The device is used as a delivery system and may be implanted by standard or minimally invasive implantation techniques. In one embodiment, the device of the present invention may be delivered by subcutaneous implantation. This may be accomplished by using aseptic techniques to surgically implant the device in any suitable formulation into any site in need of angiogenesis. Sustained release can be achieved by incorporating the active ingredients into a special carrier such as a hydrogel. Typically, a hydrogel is a network of high molecular weight biocompatible polymers, which can swell in water to form a gel like material. Hydrogels are generally known in the art. For example, hydrogels made of polyethylene glycols, or collagen, or poly(glycolic-co-L-lactic acid) are suitable for this invention. See, e.g., Phillips, et. al., J. Pharmceut. Sci. 73:1718-1720 (1984). In yet another embodiment, the device may be formed in situ by for example a needle or catheter delivering the various components that form the ECM and preferably the protein.

In another embodiment, the device can be administered topically. Topical formulations are generally known in the art and include creams, gels, ointments, lotions, powders, pastes, suspensions, sprays, and aerosols. Typically, topical formulations include one or more thickening agents, humectants, an/or emollients including but not limited to xanthan gum, petrolatum, beeswax, or polyethylene glycol, sorbitol, mineral oil, lanolin, squalene, and the like. A special form of topical administration is delivery by a transdermal patch. Methods for preparing transdermal patches are disclosed, e.g., in Brown, et al., Annual Review of Medicine. 39:221-229 (1988), which is incorporated herein by reference. Thus, the device may be delivered to exposed wounds either alone or in composite with a bandage.

In yet another embodiment, the device is delivered to the patient parenterally, i.e., intravenously or intramuscularly. For parenteral administration, the device can be formulated into solutions or suspensions, or in lyophilized forms for conversion into solutions or suspensions before use. Sterile water, physiological salime, e.g., phosphate buffered saline (PBS) can be used conveniently as the pharmaceutically acceptable carriers or diluents. Conventional solvents, surfactants, stabilizers, buffers, anti-bacteria agents, and antioxidants can all be used in the parenteral formulations, including but not limited to acetates, citrates or phosphate buffers, sodium chloride, dextrose, fixed oils, glycerine, polyethylene glycol, propylene glycol, benzyl alcohol, methyl parabens, ascorbic, acid, sodium bisulfite, and the like. The parenteral formulation can be stored in any conventional containers such as vials, ampoules, and syringes. The device of the present invention can therefore be injected into a site in need of vascularization.

Alternatively, the device may be delivered to a site in need of angiogenesis via a catheter. Catheters having spaced-apart or helical balloons for expansion within the lumen of a blood vessel and delivery of a therapeutic agent to the resulting isolated treatment site are described in U.S. Pat. Nos. 5,279,546; 5,226,888; 5,181,911; 4,824,436; and 4,636,195. A drug delivery catheter is commercially available under the trade name Dispatch™, from SciMed Life Systems, Inc., Maple Grove, Minn. Non-balloon drug deliver catheters are described in U.S. Pat. Nos. 5,180,366; 5,112,305; and 5,021,044; and PCT Publication WO 92/11890 and may be used to deliver the device of the present invention.

The active compounds can also be delivered orally in enclosed gelatin capsules or compressed tablets. Capsules and tablets can be prepared in any conventional techniques. For example, the device can be incorporated into a formulation which includes pharmaceutically acceptable carriers such as excipients (e.g., starch, lactose), binders (e.g., gelatin, cellulose, gum), disintegrating agents (e.g., alginate, Primogel, and corn starch), lubricants (e.g., magnesium stearate, silicon dioxide), and sweetening or flavoring agents (e.g., glucose, sucrose, saccharin, methyl salicylate, and peppermint). Various coatings can also be prepared for the capsules and tablets to modify the flavors, tastes, colors, and shapes of the capsules and tablets.

The device may be comprised of an ECM comprising heparin, fibronectin, and VEGF alone, or encased within or attached to a number of cormmercially available materials, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates, beads, flakes, pellets, gelatin, and cellophane (cellulose with a plasticizer). Additionally, the angiogenesis enhancing device may be incorporated into polymeric microspheres. In one embodiment, the device for implantation is made of biocompatible degradable or non-degradable polymers, i.e. polyglycolides, polyanhydrides, polyethylene vinyl acetate, and co-polymers of lactides/glycolides in various ratios. In a preferred embodiment, the ECM is attached to a bandage, preferably the bandage contains a hydrophilic coating.

In one embodiment, calcium alginate and certain other polymers that can form ionic hydrogels which are malleable are used to encase the device. For example, a hydrogel can be produced by cross-linking the anionic salt of alginic acid, a carbohydrate polymer isolated from seaweed, with calcium cations, whose strength increases with either increasing concentrations of calcium ions or alginate. The alginate solution is mixed with the device to be implanted to form an alginate suspension which is injected directly into a patient prior to hardening of the suspension. The suspension then hardens over a short period of time due to the presence in vivo of physiological concentrations of calcium ions. Modified alginate derivatives, for example, more rapidly degradable or which are derivatized with hydrophobic, water-labile chains, e.g., oligomers of ε-caprolactone, may be synthesized which have an improved ability to form hydrogels. Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Additional examples of materials which can be used to form a hydrogel include polyphosphazines and polyacrylates, which are crosslinked jonically, or block copolymers such as Pluronics™ or Tetronics™, polyethylene oxide-polypropylene glycol block copolymers which are crosslinked by temperature or pH, respectively.

Covalently crosslinkable hydrogel precursors also are useful. For example, a water soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water soluble polymer also may be utilized.

Alternatively, polymers may be utilized which include substituents which are crosslinked by a radical reaction upon contact with a radical initiator. For example, polymers including ethylenically unsaturated groups which can be photochemically crosslinked may be utilized, as disclosed in WO 93/17669. Additionally, water soluble polymers which include cinnamoyl groups which may be photochemically crosslinked may be utilized, as disclosed in Matsuda et al., ASAID Trans., 38:154-157 (1992).

In a preferred embodiment, such devices are implantable and biodegradable over a period of time equal to or less than the expected period of treatment.

### EXAMPLES

Methods

Materials

Human recombinant VEGF₁₆₅ was obtained from R&D systems (Minneapolis, Minn.). Human recombinant VEGF₁₂₁ was from Reliatech (Braunschweig, Germany). Heparinase III from flavobacterium heparinunm was a generous gift from Biomarin Pharmaceuticals (Montreal, Canada). Heparin, phenylmethylsulfonyl fluoride, sodium orthovanadate, and secondary antibody raised against rabbits and conjugated with horseradish peroxidase were obtained from Sigma (St. Louis, Mo.). ¹²⁵I -Bolton Hunter reagent was obtained from Perkin Elmer (Boston, Mass.). Phosphate buffered saline (PBS), Dulbecco.s Modified Eagle Medium (DMEM), F-12 HAM medium, peniciliinistreptomycin, L-glutamine, and 1 M HEPES buffer were from Life Technologies (Rockville, Md.). Fetal Bovine Serum (FBS) and Calf Serum (CS) were from Hyclone (Logan, Utah). Primary antibody for phospho-Erk½ was purchased from. New England Biolabs (Beverly, Mass.). Primary antibody for total Erk½ was obtained from Upstate Biotechnology (Lake Placid, N.Y.). ECL detection kit, heparin-Sepharose CL-6B and Sepharose CL-6B was purchased from Amersham (Uppsala, Sweden). Heparinase II and Heparinase I from Flavobacterium heparinum were from Seikagaku America, Inc. (Ijamsville, Md.). Collagen I was purchased from Becton Dickinson (Bedford, Mass.). ¹²⁵I-VEGF₁₆₅ and ¹²⁵I-VEGF₁₂₁ were prepared by a modified Bolton-Hunter procedure and retained their ability to stimulate endothelial cells (25).

Cell Culture

Bovine aortic endothelial cells (BAEC) were a gift from Dr. Elazer Edelman at MIT (Cambridge, Mass.). Chinese hamster ovary (CHO-K1) cells were from Dr. Jeffrey Esko at University of Alabama Birmingham (Birmingham, Ala.). NIH-3T3 cells and NIH-3T3 cells expressing VEGFR-2 (3T3/FLK) generated by retroviral infection were obtained from Dr. Nader Rahimi at Boston University (Boston, Mass.). BAEC were maintained in low glucose DMEM supplemented with 10% calf serum, 5 mM glutamine, 0.1 units/mL penicillin G and 0.1 μg/mL streptomycin sulfate. CHO-K1 cells were maintained in F-12 HAM supplemented with 5% bovine serum and 1% penicillin/streptomycin. NIH-3T3 cells were maintained in low glucose DMEM supplemented with 10% fetal bovine serum, 5 mM glutamine, 0.1 units/mL penicillin G and 0.1 μg/mL streptomycin sulfate. For experiments, BAEC were used at confluence from passages 8-16. For experiments, NIH-3T3 cells were used at subconfluence from passages 4-15. CHO-K1 cells were used at confluence. Cell number was determined with a Coulter Counter (Miami, Fla.).

Radiolabeling of VEGF.

¹²⁵I-VEGP₁₆₅ and ¹²⁵I-VEGF₁₂₁ were prepared by a modified Bolton-Hunter procedure (33). Lyophilized VEGF was dissolved in 100 mM sodium phosphate buffer, pH 8.5 (final concentration of 285 μg/mL). An aliquot (30 μL) was added to dry Bolton-Hunter reagent (1 mCi, 0.6 μmol) and incubated on ice for 2.5 h. The reaction was quenched by adding 200 μL of 0.2 M glycine and incubating on ice for 45 min. Twenty microliters of 10 mg/mL BSA in PBS and 250 μL of 1 mg/mL BSA in PBS were added. The sample was applied to a PD-10 column equilibrated and run in PBS containing 1 mg/mL BSA to separate unincorporated radiolabel from the radiolabeled VEGF. SDS-PAGE and autoradiography revealed ¹²⁵I-VEGF¹⁶⁵ and ¹²⁵I-VEGF₁₂₁ at ˜45 kDa and ˜35 kDa respectively. After radiolabeling, ¹²⁵I-VEGF₁₆₅ retained its ability to bind to a heparin-Sepharose column and was eluted with high concentrations of salt. Also, both ¹²⁵I-VEGF₁₆₅ and ¹²⁵I-VEGF₁₂₁ were able to stimulate activation of Erk½ on BAEC, indicating that the radiolabeling procedure did not adversely disrupt VEGF structure.

¹²⁵I-VEGF Binding

Equilibrium binding assays were carried out with confluent cell cultures. BAEC were seeded at 75,000 cells/well in 24-well dishes (Corning Inc, Corning, N.Y.). CHO cells were seeded at 100,000 cells/well in 24-well dishes. Cells were grown for 24 hours. Binding assays conducted at various pHs were carried out in binding buffer consisting of 25 mM HEPES adjusted to the indicated pH (7.5-5.5) in DMEM (without bicarbonate) containing 0.1% BSA. Cells were washed once with ice cold binding buffer. Binding buffer was added to cells and incubated at 4° C. for 10 min. ¹²⁵I-VEGF₁₆₅ (0.12 nM) or ¹²⁵I-VEGF₁₂₁ (0.14 nM) was added to cells. Cells were incubated for 2.5 h at 4° C. After the binding period, unbound ¹²⁵I-VEGF was removed by washing the cells 3 times with ice cold binding buffer. To dissociate VEGF interactions involving HSPG, cells were exposed to a high salt buffer (25 mM HEPES, pH 7.5, 2 M NaCl) for approximately 5 s and then rinsed with PBS. Cells were then solubilized with 1 N NaOH to account for the remaining interactions that are presumably VEGF bound to receptor. It has been shown that FGF-2 can be dissociated from HSPG on cell surfaces by using a 2 M NaCl wash (34).

Equilibrium binding assays were carried out on fibronectin, collagen I, or BSA coated dishes. Binding assays conducted at various pHs were carried out in binding buffer containing 25 mM HEPES adjusted to the indicated pH (7.5-5.5) in DMEM (without bicarbonate) containing 0.1% BSA. Matrices were washed once with ice cold binding buffer. Binding buffer was added to the matrices and incubated at 4° C. for 10 min. ¹²⁵I-VEGF₁₆₅ (0.12 nM) or ¹²⁵I-VEGF₁₂₁ (0.14 nM) was added to matrices and the binding reaction was allowed to proceed for 2.5 h at 4° C. After the binding period, unbound ¹²⁵I-VEGF was removed by washing the matrices 3 times with ice cold binding buffer. Matrices were then solubilized with 1 N NaOH to extract the bound ¹²⁵I-VEGF. ¹²⁵I-VEGF binding was quantified by counting in a Cobra Auto-Gamma 5005 γ-counter (Packard Instruments, Meridian, Conn.). Nonspecific binding was measured using a 500-fold excess of unlabeled VEGF and this value was subtracted from each sample. To determine the effects of removing heparan sulfate proteoglycans, fibronectin-coated plates were treated with 0.5 units/mL of heparinase III for 1 h at 37° C. prior to conducting the binding studies. Heparinase III and digestion products were removed by washing 2 times with binding buffer. To determine the effects of heparin, various concentrations were added to the binding buffer prior to the addition of ¹²⁵I-VEGF. All conditions were conducted in triplicate and each experiment was repeated at least 3 separate times. It has been established that VEGF elutes from a heparin-Sepharose column with 0.69 M NaCl (35). Consistent with these findings, we have established that 0.75 mM NaCl is enough to sufficiently remove VEGF from the first binding fraction. Moreover, increasing the ionic strength up to 2 M NaCl did not remove additional VEGF. Therefore, we conclude that the first wash contains VEGF that is able to interact with HSPG through ionic interactions and that the remaining VEGF-receptor interactions are observed by solubilizing the cells in 1 N NaOH. ¹²⁵I-VEGF binding was quantified by counting in a Cobra Auto-Gamma 5005 γ-counter (Packard Instruments, Meridian, Conn.). Nonspecific binding was measured using a 500-fold excess of unlabeled VEGF and the calculated value was subtracted from each sample. Replicate cells were maintained under the same conditions as sample cells in the absence of ¹²⁵I-VEGF and were used to measure final experimental pH. It was found that the pH of the various buffers did not change during the course of the binding experiments. Also, cell numbers were determined after the binding incubation and did not vary between different pHs. In addition, toxicity experiments were conducted in cells maintained in the various pH buffers for up to 4 h at 37° C. Cell number and viability (trypan blue exclusion) did not vary over the 4 h incubation across the pH range of 7.5-5.0. To determine the effects of removing heparan sulfate proteoglycans, cells were treated with 0.5 units/mL of heparinase III for 1 h at 37° C. prior to conducting the binding studies. Heparinase and digestion products were removed by washing 2 times with binding buffer. To determine the effects of heparin, various concentrations were added to cells prior to the addition of ¹²⁵I-VEGF. All conditions were conducted in triplicate and each experiment was repeated at least 3 separate times.

Dissociation of ¹²⁵I-VEGF from Fibronectin

Fibronectin-coated dishes were prepared and binding assays were conducted at pH 7.5 or pH 5.5 as described. ¹²⁵I-VEGF₁₆₅ (0.6 nM) or ¹²⁵I-VEGF₁₂₁ (0.7 nM) was added to fibronectin for 2.5 h at 4° C. After the binding incubation, unbound VEGF was removed by washing the fibronectin-coated dishes three times with binding buffer. Fresh binding buffer (pH 7.5, 6.5, 5.5) was added (500 μL/well) and the plates were allowed to incubate at 4° C. At various time points, the buffer containing released VEGF was collected, the wells were washed once with 500 μL of the corresponding buffer, fresh buffer was added, and the incubation continued. After the final time point, the fibronectin was solubilized in 1 N NaOH to account for the remaining ¹²⁵I-VEGF bound. The effect of heparin was also evaluated on VEGF dissociation from fibronectin. Heparin (1 μg/mL) was added to fibronectin prior to the addition of¹²⁵I-VEGF. After the binding incubation, fresh buffer without heparin was added to the well.

Preparation of ECM-Coated Dishes

ECM-coated dishes were prepared as previously described (33,36,37). BAEC were plated at 25,000 cells per well in 24-well dishes. Cells were grown for 3 days and reached confluence. The cells were lysed by incubating the cultures for 3 min at 23° C. in a solution containing 0.5% Triton X-100, 20 mM NH4OH in phosphate buffered saline, leaving the ECM associated with the culture surface. Subsequently the ECM was washed 4 times with PBS. The ECM remained intact and was characterized to contain HSPG by 35S labeling of HS chains (data not shown). Non-treated tissue culture dishes were coated with either fibronectin, collagen I, or bovine serum albumin (BSA) at 20 μg/mL in ion free PBS overnight at 4° C. To maintain consistent coating between dishes, 250 μL per 1 cm² was used to coat the dishes (31). Protein assays reveal ˜70% adsorption of proteins under these conditions.

Heparin-Sepharose Columns

Heparin-Sepharose affinity chromatography was used to assess VEGF₁₆₅ and VEGF₁₂₁ binding to heparin directly (35). ¹²⁵I-VEGF₁₆₅ (0.01 μM), ¹²⁵I-VEGF₁₂₁ (0.011 μM) or ¹²⁵I-EGF (0.05 μM) was incubated in column buffers (150 mM NaCl, 25 mM HEPES pH 7.5 or pH 5.5) for 15 min. Columns (1 mL packed with heparin-Sepharose CL-6B) were equilibrated with the column buffers corresponding to those that the growth factor (GF) was incubated in by passing 3 mL of buffer through the columns. Individual columns were prepared for each sample condition. After the 15 min incubation, the 125I-GF samples were applied to the columns and the column was washed with the same buffer (1 mL) in which it was equilibrated. Flow through was collected. The ¹²⁵I-GF was then eluted with either the same buffer in which it was incubated or the other pH buffer. This eluant was collected and labeled pH Wash. Fractions were TCA precipitated to remove any free ¹²⁵I from ¹²⁵I-GF. Samples were quantitated in a γ counter. The fraction of ¹²⁵I-GF bound to the column was quantified by counting the entire column in a γ counter.

Activation of Erk½

BAEC were plated at 20,000 cells per well in 6-well dishes. After 24 h, the medium was replaced with DMEM containing 0.5% CS for 24 h, to quiesce the cells. Prior to stimulation with VEGF, cells were washed with binding buffer at pH 7.5, 6.5 or 5.5 and remained in the binding buffer for 90 min. VEGF₁₆₅ (0.6 nM) or VEGF₁₂₁ (0.7 nM) was added to the cells at various time points. Binding buffer with VEGF was removed and cells were extracted in 0.1% Triton X-100, 150 mM NaCl, 10 mM Tris, pH 7.5, 1 mM EDTA, 1 mM EGTA, 0.5% NP-40 containing 1 mM phenylmethylsulfonyl fluoride and 0.2 mM sodium orthovanadate. Cell lysates were spun at 13,000 μg for 10 min at 4° C. Supernatants were collected. BCA protein assays were conducted to determine total protein content. An equal amount of protein from each sample was subjected to SDS-PAGE (12% gel) and transferred to mnobilon membranes (Millipore Corp., Bedford, Mass.). Membranes were blocked with 5% BSA in tris buffered.saline with 0.05% Tween-20. Subsequently, the membranes were incubated with anti-phospho-Erk½ or anti-Erk½. Immunoreactive bands were visualized with chemiluminescence using horseradish peroxidaseconjugated anti-rabbit IgG and ECL reagent. Membranes were stained with Ponceau S to evaluate total protein loaded. Experiments were repeated for NIHJ3T3 cells plated at 50,000 cells per well in 6-well dishes.

### Example 1

### Affect of Heparin and Heparin-Derived Compounds on Binding

Results

VEGF₁₆₅ and VEGF₁₂₁ Binding to Cell Surfaces are Altered by pH

At sites of angiogenesis, such as tumors and wounds, the environment is rather hypoxic. Hypoxia increases expression of VEGF thereby promoting increased rates of migration and proliferation of endothelial cells. Hypoxic environments also lead to decreases in extracellular pH. While the intracellular signaling events occurring under hypoxic conditions in response to VEGF have been of major focus, there has been little attention to how acidity affects VEGF outside of cells. Therefore, to investigate the role of extracellular pH on VEGF₁₆₅ and VEGF₁₂₁ interactions with cell surfaces, binding assays were conducted with confluent BAEC at various pHs ranging from pH 7.5-5.5 (FIG. 1). It was found that as pH decreased, VEGF₁₆₅ and VEGF₁₂₁ binding to BAEC increased dramatically. HSPG-mediated VEGF₁₆₅ binding at pH 5.5 was ˜2.5 fold greater than that at pH 7.5 while VEGF₁₂₁ binding increased 20 fold. At pH 7.5, only ˜40% of the total bound VEGF₁₆₅ bound through the HSPG component, where at pH 5.5, greater than 50% of the total VEGF₁₆₅ bound occurred through the HSPG component. For VEGF₁₂₁, there appeared to be a more dramatic specificity for binding to the HSPG component at the lower pHs. At pH 7.5, ˜40% of total bound VEGF₁₂₁ occurred through the HSPG component, while at pH 5.5, ˜80% bound through the HSPG component. Experiments were repeated on CHO-K1 cells, which are a cell type that do not express endogenous VEGF receptors. Similar results were found where decreases in extracellular pH increased VEGF binding to these cells (FIG. 2). We also observed similar results when these experiments were conducted on NIH-3T3 cells (data not shown). These results suggest that the increased VEGF binding at acidic pH does not depend on VEGF receptors.

To determine if HSPG play a role in the pH-induced changes in cell surface binding, endothelial cells were treated with heparinase III to degrade heparan sulfate chains prior to the addition of VEGF. We found that heparinase III treatment reduced VEGF₁₆₅ and VEGF₁₂₁ binding under neutral (pH 7.5) and acidic (pH 5.5) conditions by ˜60% and ˜20% respectively (data not shown). Thus, HSPG are involved in VEGF₁₆₅ and VEGF₁₂₁ interactions with endothelial cell surfaces at neutral and acidic pH.

Changes in Extracellular pH Alter VEGF₁₆₅ and VEGF₁₂₁ Interactions with Extracellular Matrices

HSPG are a major component of extracellular matrices. Since the previous results suggest that the acidic-pH mediated binding of VEGF may be independent of VEGF receptors, we wanted to determine if binding of VEGF to extracellular matrix would be affected by changes in pH. To investigate the role of pH on VEGF interactions within the extracellular matrix, VEGF binding to BAEC-deposited extracellular matrices was characterized. BAEC were grown for 3 days until confluent. Cell layers were extracted leaving BAEC-deposited extracellular matrix coated dishes. Extracellular matrices were labeled with ³⁵SO₄ to determine if HSPG are a component of these matrices. Matrices were treated with heparinase III after labeling and it was found that ³⁵S radioactivity decreased by 66% after heparinase III treatment, confirming that HSPG were a component of the matrices. Matrices were washed with cold binding buffer at various pHs (7.5, 7.0, 6.5, 6.0, 5.5) and ¹²⁵I-VEGF₁₆₅ and ¹²⁵I-VEGF₁₂₁ binding were measured (FIGS. 3A & 3B). It was observed that as pH decreased, VEGF₁₆₅ and VEGF₁₂₁ binding increased by ˜5 fold. At pH 5.5, heparinase III treatment decreased VEGF₁₆₅ and VEGF₁₂₁ binding to the BAEC-deposited matrices by ˜30% indicating that HSPG contribute to the acidic-pH mediated binding of VEGF to ECM (data not shown). Together, these observations suggest that interactions with the ECM are a major component of the acidic-pH mediated increase in VEGF binding observed with whole cells.

pH Alters the Affinity of VEGF₁₆₅ and VEGF₁₂₁ for Heparin

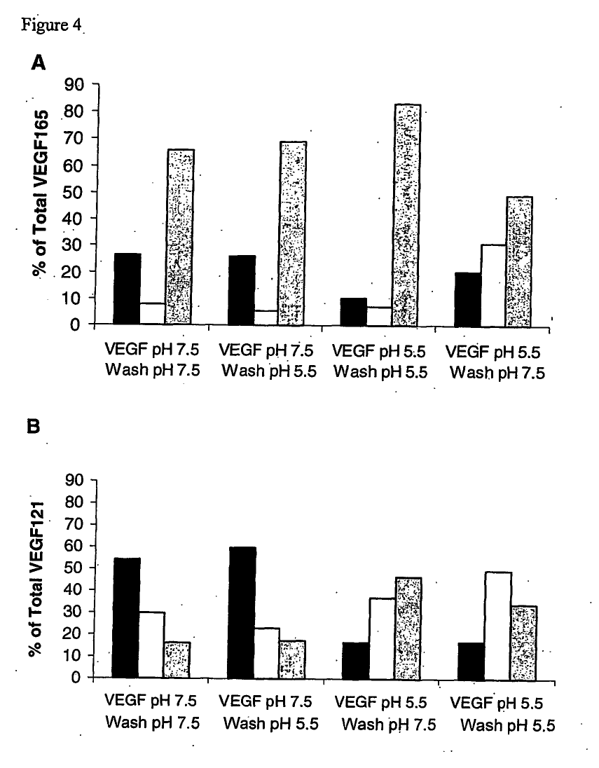

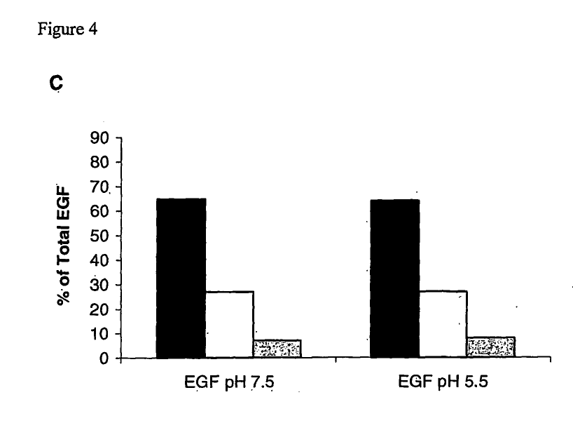

The increased binding of VEGF to endothelial cells and the extracellular matrix at low pH suggests that VEGF interactions with heparan sulfate are enhanced under acidic conditions. To assess how pH directly affects VEGF-HS interactions, VEGF₁₆₅ and VEGF₁₂₁ binding to heparin-Sepharose was analyzed at pH 7.5 and pH 5.5 (FIGS. 4A and 4B). VEGF₁₆₅ and VEGF₁₂₁ were incubated in pH 7.5 or pH 5.5 binding buffer for 15 min. Heparin-Sepharose columns were equilibrated in the pH solution in which the VEGF was incubated. VEGF was applied to the column and then eluted with either the pH 7.5 or pH 5.5 solutions. It was observed that 65% of VEGF₁₆₅ bound to the column at pH 7.5 and ₈₀% bound at pH 5.5. Also, when VEGF₁₆₅ was incubated and applied to the column at pH 5.5 and subjected to a pH 7.5 wash, 30% of the bound VEGF₁₆₅ was released. This data suggests that VEGF₁₆₅ has a higher affinity for heparin at pH 5.5 and that these interactions are reversible when returned to neutral pH. Only 20% of VEGF₁₂₁ was bound to the column at pH 7.5, consistent with earlier observations that this isoform does not show significant heparin afnnity. However, at pH 5.5, 45% of the applied VEGF₁₂₁ was bound. Furthermore, when VEGF₁₂₁ was bound to the column at pH 5.5 and then washed with pH 7.5, 50% of the bound VEGF₁₂₁ was released from the column. This data suggests that acidic conditions can reversibly convert VEGF₁₂₁ to a heparin-binding protein. As a control, VEGF₁₆₅ and VEGF₁₂₁ were passed through Sepharose CL-6B columns to ensure that they were interacting with the heparin and not the Sepharose at low pH. It was found that both VEGF₁₆₅ and VEGF₁₂₁ passed freely through Sepharose CL-6B at pH 7.5 and pH 5.5 (data not shown). Moreover, to conclude that pH does not have some general non-specific effects on protein adsorption to heparin-Sepharose, EGF, a non-heparin binding growth factor, was eluted through hepanrn-Sepharose columns at pH 7.5 and pH 5.5 (FIG. 4C). EGF did not show increased binding to the column at acidic pH. Therefore, increased binding of VEGF₁₆₅ and VEGF₁₂₁ to heparin at acidic pH is not a general phenomenon for all proteins.

Exogenous Heparin Inhibits VEGF Binding at Acidic pH

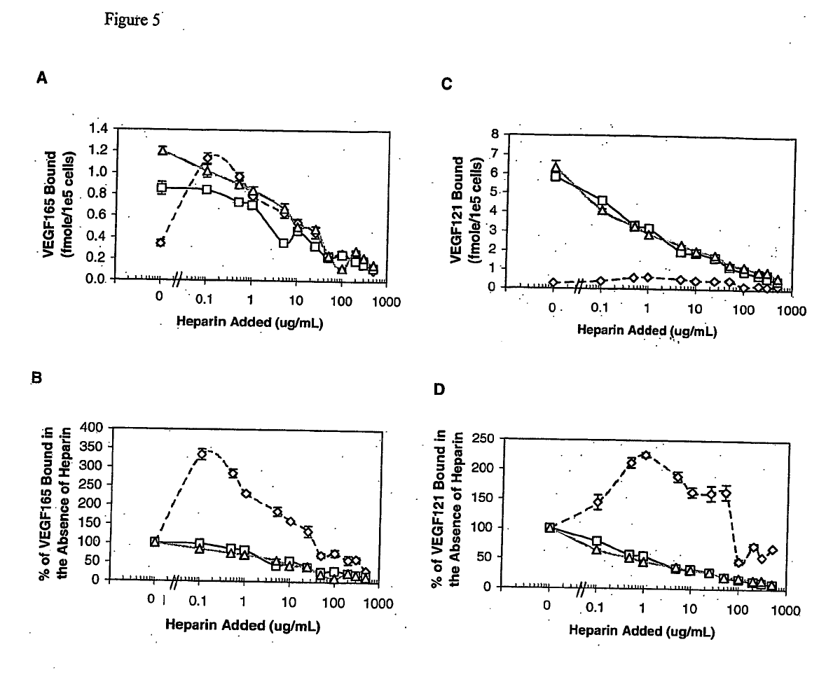

HSPG have been shown to play a role in modulating VEGF activity. Here we show that VEGF-HS interactions were altered by pH. Thus, we wanted to determine how the addition of heparin would affect VEGF₁₆₅ and VEGF₁₂₁ binding to BAEC at various pHs (FIG. 5). Binding assays were conducted at pH 7.5, 6.5 and 5.5. Various concentrations of heparin (0.1-500 μg/mL) were added to cells prior to the addition of ¹²⁵I-VEGF, and cell surface binding was determined. It was found that VEGF₁₆₅ binding to BAEC at the three pHs was inhibited at high concentrations of heparin (50-500 μg/mL) (FIG. 5A). At pH 7.5, low concentrations of heparin (0.1-25 μg/mL) potentiated VEGF₁₆₅ binding significantly (˜3 fold). Interestingly, 0.1 μg/mL of heparin increased VEGF₁₆₅ binding at pH 7.5 to levels that were equivalent to those at pH 5.5 in the absence of heparin. At pH 6.5, 0.1 μg/mL of heparin maintained the same amount of VEGF₁₆₅ bound as VEGF₁₆₅ bound in the absence of heparin. At pH 5.5, 0.1 μg/mL of heparin decreased VEGF₁₆₅ binding. Interestingly, the trends for VEGF₁₆₅ binding at pH 7.5, 6.5, and 5.5 in the presence of 1-500 μg/mL heparin were almost superimposable. It appears that low concentrations of heparin had the same affect on VEGF₁₆₅ binding as did reduced pH. However, the effect of heparin on VEGF₁₂₁ binding at pH 7.5, 6.5, and 5.5were quite different (FIGS. 5C and D). It was observed that all concentrations of heparin decreased VEGF₁₂₁ binding at pH 6.5 and 5.5. At pH 7.5, low concentrations of heparin (0.1-50 μg/mL) were able to potentiate VEGF₁₂₁ binding (FIG. 5D), however the potentiation was unable to reach levels of VEGF₁₂₁ binding at pH 6.5 or pH 5.5 in the absence of heparin. Interestingly, there appeared to be a shift in the amount of heparin required to potentiate VEGF₁₂₁ binding in relation to VEGF₁₆₅ binding under neutral conditions. Maximal VEGF₁₆₅ binding occurred in the presence of 0.1 μg/mL of heparin while maximal VEGF₁₂₁ binding occurred at 1 □g/mL of heparin (FIGS. 5B and D). Therefore, heparin and pH may coordinate to regulate VEGF activity.

Erk½ Activation by VEGF is Affected by Decreases in Extracellularp pH

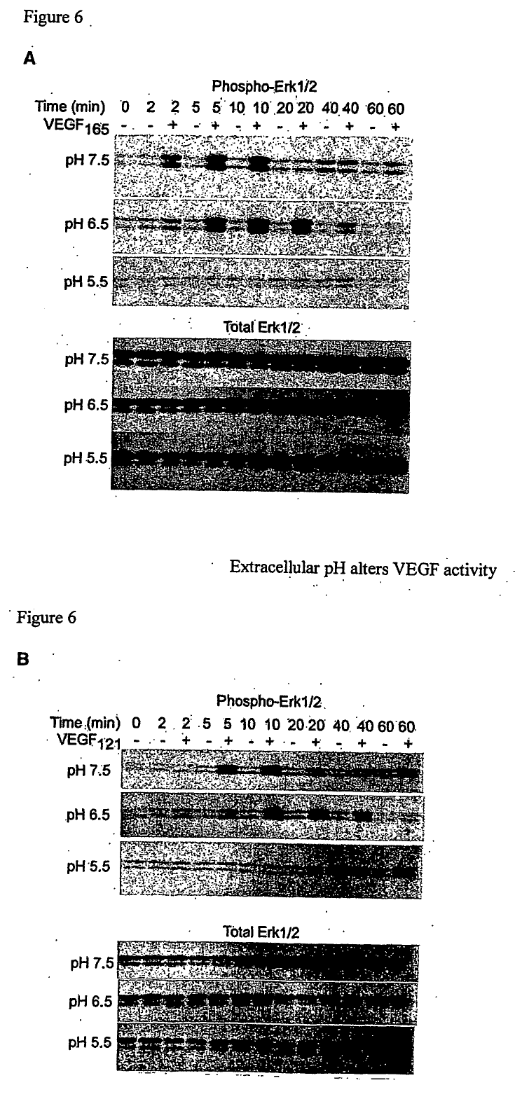

VEGF stimulates a number of signaling molecules. One that has been well characterized in response to VEGF is activation of the extracellular signal-regulated kinases (Erk½) (38). Since extracellular pH modulates VEGF interactions with BAE cell surfaces, we wanted to determine how these differences might relate to activation of Erk½. Quiescent BAEC at various pHs were stimulated with VEGF and the activation of Erk½ was analyzed by Western blot (FIG. 6). At pH 7.5, VEGF₁₆₅ stimulated Erk½ activation with a peak activation time of 5 min. At pH 6.5, Erk½ activation peaked at 10 min. At pH 5.5, Erk½ did not appear to be phosphorylated in response to VEGF₁₆₅ (FIG. 6A). Total Erk½ was not affected by pH. VEGF₁₂₁ displayed a similar pattern of Erk½ activation as that of VEGF₁₆₅ (FIG. 6B). Cell number was determined under each condition and was identical at the three different pHs tested. These experiments were repeated on NIH-3T3 cells and NIH-3T3 cells transfected with VEGFR-2. It was found that VEGF₁₆₅ stimulated Erk½ phosphorylation in the VEGFR-2 expressing cells at pH 7.5 but was unable to stimulate activation at pH 5.5. VEGF₁₆₅ was unable to activate Erk½ at pH 7.5 or 5.5 in the parent NIH-3T3 cells (data not shown).

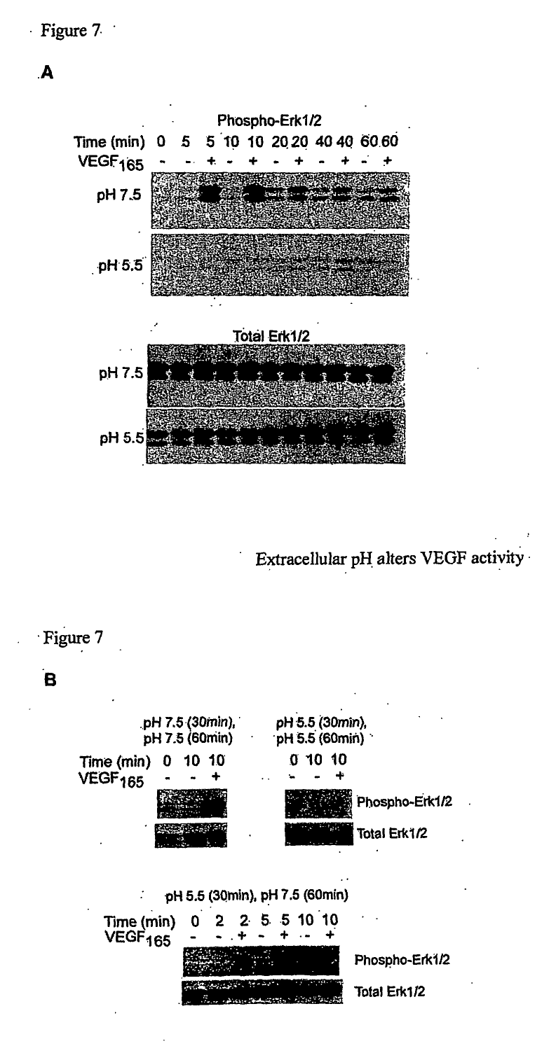

To ensure that the acidic pH (5.5) did not damage VEGF in such a way as to eliminate activity, experiments were conducted where VEGF₁₆₅ was incubated at pH 5.5 for 20 min prior to the addition of cells at pH 7.5 (FIG. 7A). Pre-incubating VEGF₁₆₅ at acidic pH showed similar Erk½ activation as untreated VEGF₁₆₅ with a peak activation time of 5 min. Therefore, the pH affects on VEGF₁₆₅ activity were reversible. Also, to ensure that BAEC activity could be recovered after being exposed to acidic pH, experiments were conducted where BAEC were incubated at pH 5.5 for 30 min at 37° C. followed by pH 7.5 incubation for 60 min, and then stimulated with VEGF₁₆₅ for 2, 5, and 10 min (FIG. 7B). It was found that cells could be stimulated in response to VEGF₁₆₅ to the same extent as cells that were not exposed to acidic pH. Thus, the pH affects on VEGF₁₆₅ and BAEC activity were reversible.

Conclusions

Low extracellular pH is a common feature of solid tumors. Endothelial cells are exposed to this environment while undergoing angiogenesis under many pathological and physiological conditions. We have discovered that varying pH has an impact on extracellular proteins, which ultimately can influence cell activity. Forsten et al. discovered that low extracellular pH increases cell surface binding and nuclear localization of IGF-1 (39). Recently, Wahl et al. found that under acidic conditions, angiostatin decreased endothelial cell migration and increased cell death (40). However, D. Arcengelo et al. found that endothelial cells are protected from apoptosis in an acidic environment (32). These results provide evidence for the importance of pH in biological systems. Our results demonstrate that a consequence of decreased local pH is an increase in cell surface and ECM binding of VEGF₁₆₅ and VEGF₁₂₁. The increased VEGF binding at low pH was disruptable by high salt and neutral pH. Thus, acidic conditions appear to alter VEGF and/or VEGF binding sites reversibly, indicating that VEGF deposited within hypoxic and acidic regions of tissues would be rapidly released as the pH increases, where it could stimulate nearby endothelial cells.

The mechanism by which acidic pH increases VEGF binding to cells and ECM appears to involve increased binding to HSPG and is not dependent on VEGF receptors. The acidic pH increase in binding was observed with VEGFR-deficient cells, CHO and NIH-3T3, as well as with acellular ECM. Moreover, the increased binding to cells and ECM was reduced by pre-treatment with the heparan sulfate degrading enzyme heparinase III, indicating that HSPG are involved in this process. The increased interaction of VEGF with HSPG at low pH does not likely involve. the traditional heparin-binding domain on VEGF₁₆₅ since increased binding is observed with VEGF₁₂₁ as well. Thus, reduced pH could stabilize VEGF structure for optimal interaction, or may create new heparin-binding sites within VEGF. Indeed, within the shared regions of VEGF₁₂₁ and VEGF₁₆₅ sequence there are several histidine residues near basic amino acids that could comprise regions of positive charge at acidic pH.

Consistent with the model that acidic pH induces the formation of a new heparin-binding domain within amino acids 1-121 in VEGF; we observed a greater relative effect of pH on VEGF₁₂₁ binding to BAEC compared to that with VEGF₁₆₅. While, VEGF₁₆₅ binds HSPG well under neutral conditions, a new heparin-binding site under acidic conditions may enhance this existing property. In contrast, VEGF₁₂₁ does not contain the neutral pH heparin-binding domain; thus, the generation of the property to bind heparin at low pH results in ˜20 fold increased binding. This property may be conferred by the generation of a new binding site under acidic conditions, or alternatively, might result from a conformational change in the growth factor by positioning basic residues properly to bind to heparin.

The addition of exogenous heparin had different affects on VEGF₁₆₅ and VEGF₁₂₁ binding to endothelial cells. At neutral pH, low concentrations of heparin potentiated VEGF binding while high concentrations inhibited VEGF binding. This data is supported by Gitay-Goren et al., who have found that exogenous heparin potentiates VEGF binding to VEGFR-2 (16). However, at acidic pH, all concentrations of heparin inhibited VEGF binding. For VEGF₁₆₅, the addition of low concentrations of heparin enhanced VEGF₁₆₅ binding at neutral pH to levels that were equivalent to those for VEGF₁₆₅ binding at low pH. The addition of exogenous heparin had different affects on VEGF₁₂₁ binding. High concentrations of heparin inhibited VEGF₁₂₁ binding at neutral and acidic pH. Low concentrations of heparin enhanced VEGF₁₂₁ binding. However unlike VEGF₁₆₅ , heparin could not enhance VEGF₁₂₁ binding to the high levels observed at acidic pH. The differences in heparin effects on VEGF₁₆₅ and VEGF₁₂₁ binding might reflect the generation of a new heparin-binding domain in VEGF at acidic pH.

The MAPK pathway has been shown to be activated in response to VEGF in endothelial cells (38). One component of this pathway is the activation of Erk½. In this study, we utilized the activation of Erk½ as a marker of biological response to VEGF under different extracellular pHs. It appeared that VEGF-mediated Erk½ activation at pH 5.5 was reduced to basal levels. This data correlates with other results showing that proangiogenic activity is reduced under acidic pH (31,40). Moreover, exposing the cells to an acidic pH did not damage cells since cells that were returned to neutral pH could once again be stimulated by VEGF₁₆₅. Also, acidic pH did not appear to induce a permanent change in VEGF structure, in that once VEGF returned to neutral pH, it was able to stimulate Erk½ activation.

Our data shows that extracellular pH can participate in directing angiogenesis by establishing gradients of VEGF stored within the ECM. In environments where the extracellular pH is decreased, such as at sites of injury and tumors, VEGF may be sequestered in the ECM via its potential new heparin-binding site. As new vasculature is recruited to these sites the pH would increase and the stored VEGF would be released from the ECM to futer activate endothelial cells. In conclusion, we have found that extracellular pH can dramatically modulate VEGF binding to and activity in endothelial cells. Increased VEGF deposition within the ECM in hypoxic and locally acidic tissue environments might participate in guiding the growth of new blood vessels to these undervascularized regions.

### Example 2

Factors in Addition to Heparin and Heparin-Derived Compounds Affecting Binding

Activation of Erk½ in BAEC

Erk12/ activation in BAEC was evaluated in response to VEGF that had been pre-bound to BAEC at pH 5.5. BAEC were plated at 20,000 cells per well in 6-well dishes. After 24 h, the medium was replaced with DMEM containing 0.5% CS for 24 h, to quiesce the cells. Cells were incubated with binding buffer at pH 5.5 for 10 min at 37° C. VEGF₁₆₅ (0.6 nM) was added to the cells at pH 5.5 for 60 miin at 37° C. Unbound VEGF₁₆₅ was removed. Cells were incubated for 10 min in buffer at pH 7.5, 7.0, 6.5, 6.0, or 5.5. Erk½ activation in BAEC was evaluated in response to VEGF that had been dissociated from fibronectin. Quiescent endothelial cells were generated as stated above. Binding buffer (1 mL) was added to cells at pH 7.5 or pH 5.5, for 90 min. VEGF₁₆₅ (3.0 nM) and VEGF₁₂₁ (3.5 nM) were allowed to bind to fibronectin-coated dishes in the presence of 1 μg/mL of heparin for 1 h at 37° C. as described above. After binding, fibronectin-coated dishes were washed 3 times to remove any unbound VEGF. New binding buffer (1 mL) was added to the fibronectin-coated dishes at pH 7.5 or pH 5.5 for 20 min. The buffer containing released VEGF was collected and added to BAEC. Cell lysates were collected after 2, 5, 10, 20, and 30 min stimnulation. Cells were extracted in 0.1% Triton X-100, 150 mM NaCl, 10 mM Tris, pH 7.5, 1 mM EDTA, 1 mM EGTA, 0.5% NP-40, containing 1 mM phenylmethylsulfonyl fluoride and 0.2 mM sodium orthovanadate. Cell lysates were spun at 13,000×g for 10 min at 4° C. Supernatants were collected. BCA protein assays were conducted to determine total protein content. An equal amount of protein from each sample was subjected to SDS-PAGE (12% gel) and transferred to Immobilon membranes (Millipore Corp., Bedford, Mass.). Membranes were blocked with 5% BSA in tris buffered saline with 0.05% Tween-20. Subsequently, the membranes were incubated with anti-phospho-Erk½ or anti-Erk½. Blots were stripped of antibodies and reprobed with anti-Erk½ antibody. Immunoreactive bands were visualized with chemiluminescence using horseradish peroxidase-conjugated anti-rabbit IgG and ECL reagent. Membranes were stained with Ponceau S to evaluate total protein loaded. Experiments were repeated at least 3 times.

Results

Heparinase Decreases VEGF₁₆₅ and VEGF₁₂₁ Binding to BAEC

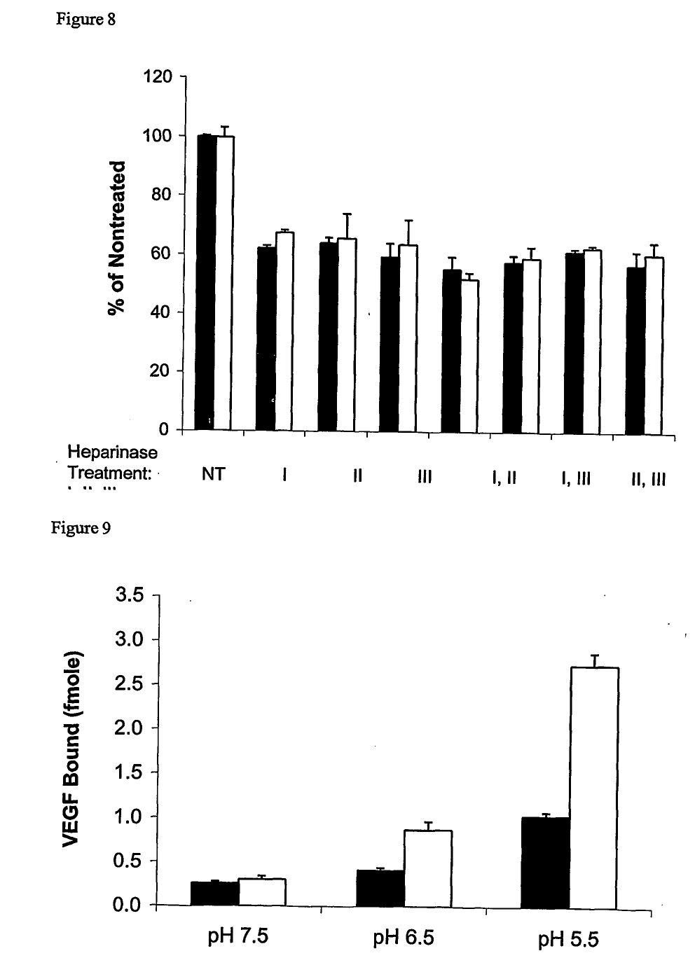

The prior Example demonstrated that VEGF₁₆₅ and VEGF₁₂₁ interactions with BAEC and ECM are enhanced by acidic pH. In addition, VEGF₁₆₅ and VEGF₁₂₁ binding to BAEC and ECM at pH 7.5 and pH 5.5 was reduced by pretreating the cells with heparinase III to digest HS chains. These results suggested that VEGF interactions with endothelial cells at acidic pH are dependent on HS. However, heparinase III treatment only reduced binding by ˜35% suggesting that other binding components exist for VEGF at reduced pH, or that heparinase III digestion did not completely remove all of the HS chains involved. We conducted binding assays on BAEC that were treated with various heparinases (FIG. 8). BAEC were treated with heparinase I, heparinase II, heparinase III, or a combination of the enzymes for 1 h at 37° C. Binding assays were conducted at pH 5.5 with VEGF₁₆₅ and VEGF₁₂₁. It was observed that heparinase I, II, and III reduced VEGF₁₆₅ and VEGF₁₂₁ binding by ˜40%. Combining the various heparinases did not further reduce binding. These results indicate that VEGF binding at acidic pH involves components in addition to HS.

VEGF Binding to Fibronectin Increases with Decreasing pH

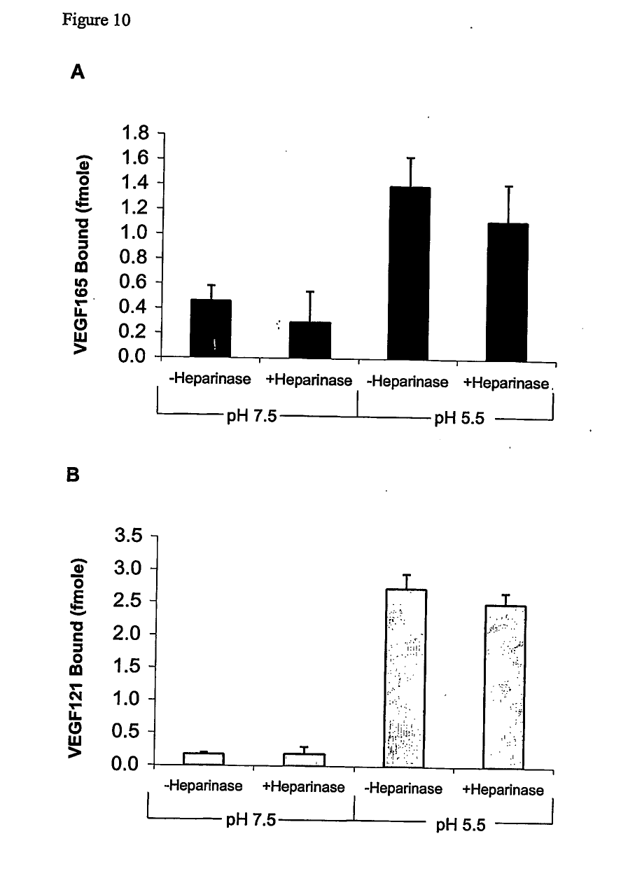

The increased binding of VEGF to cells at low pH was not dependent on the expression of VEGF receptors. VEGF directly binds to fibronectin near the N- and C-terminal domains. Therefore, altered VEGF-fibronectin interactions may be responsible for the HS-independent binding of VEGF at low pH. To test this possibility, we conducted binding assays on fibronectin-coated dishes with VEGF₁₆₅ and VEGF₁₂₁ at pH 7.5, 6.5, and 5.5 (FIG. 9). It was found that as the pH decreased, VEGF₁₆₅ and VEGF₁₂₁ binding to fibronectin increased. VEGF₁₆₅ binding at pH 5.5 was increased by ˜4-fold and VEGF₁₂₁ binding was increased by ˜9-fold compared to that at pH 7.5. Therefore, VEGF interactions with fibronectin appear to be sensitive to pH. The fibronectin used was purified from bovine plasma, and fibronectin is known to bind heparin and HS. Thus, it is possible that these preparations might contain contaminating HS, which could be responsible for the pH sensitive binding of VEGF to the fibronectin-coated dishes. To determine if endogenous HS in the fibronectin matrix is contributing to VEGF binding at acidic pH, we treated fibronectin-coated dishes with heparinase III to remove any contaminating HS chains. We observed that heparinase treatment had no effect on VEGF₁₆₅ or VEGF₁₂₁ binding to fibronectin at neutral or acidic pH (FIGS. 10A and B). Therefore, we conclude that the increased binding of VEGF to fibronectin at acidic pH is not the result of contaminating HS.

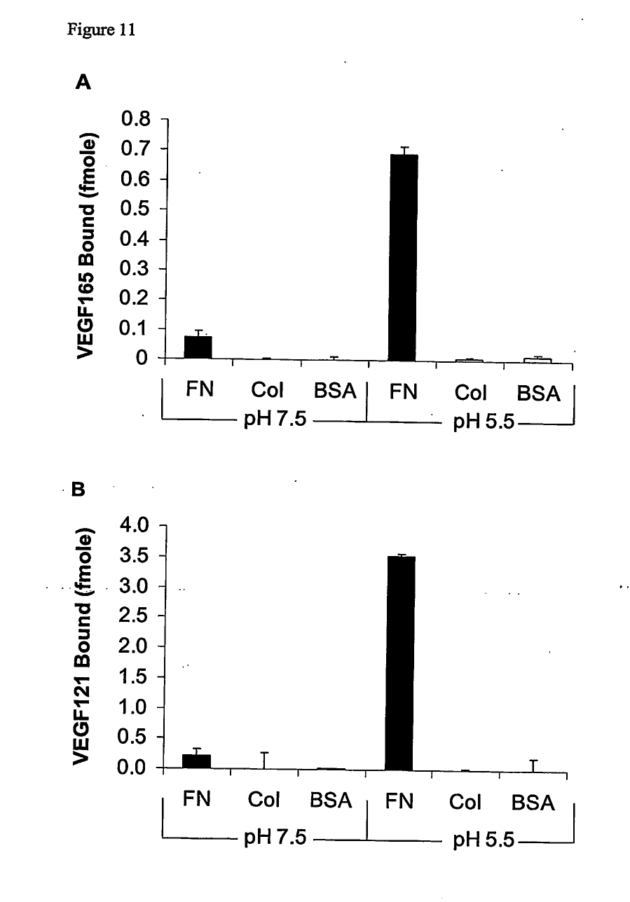

Fibronectin is just one component of the ECM. Therefore, we wanted to investigate whether the ability to bind VEGF at low pH was a general property of protein-coated surfaces or if it was specific to fibronectin. Toward this end, we conducted binding assays on collagen type I-coated dishes and bovine serum albumin (BSA)-coated dishes in comparison with binding to fibronectin-coated dishes (FIG. 11). It was observed that there was no significant binding of VEGF₁₆₅ (FIG. 11A) or VEGF₁₂₁ (FIG. 11B) to collagen type I or to BSA at pH 7.5 or pH 5.5. Therefore, the binding observed for fibronectin was not a general nonspecific interaction that occurs with all proteins absorbed to tissue culture plastic.

Heparin Potentiates VEGF Binding to Fibronectin at Acidic pH

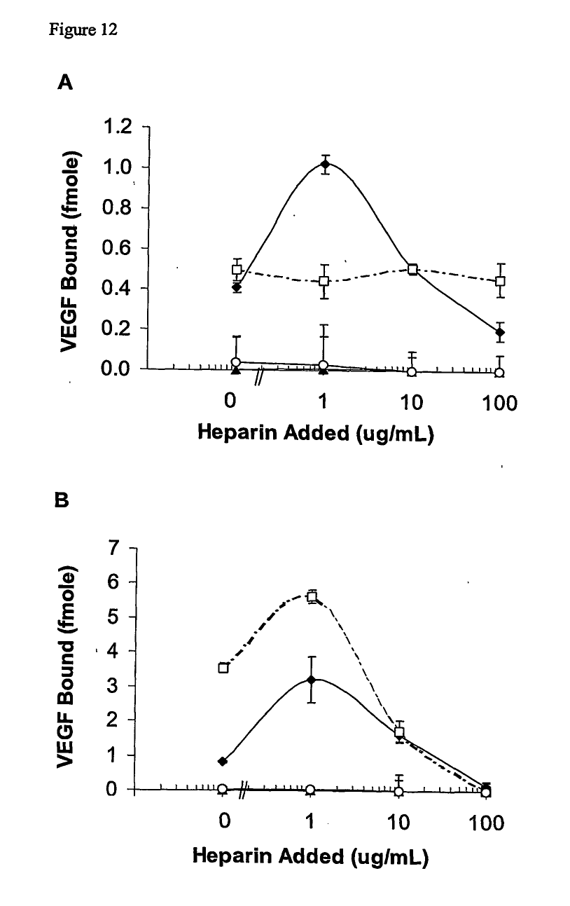

Fibronectin is a complex ECM protein with many protein-binding domains. HSPG have been found to interact with fibronectin in two domains, one near the N-terminus and the other near the C-terminus. Certain isoforms of VEGF also interact with HS. It has been shown that VEGF interactions with heparin are enhanced by decreased pH. As discussed below, VEGF-heparin-fibronectin interactions are also affected by changes in pH. Also, collagen type I interacts with HS, therefore, HS may serve to bridge VEGF to collagen type I. We conducted binding-assays with VEGF₁₆₅ and VEGF₁₂₁ on fibronectin- and collagen type I-coated dishes in the presence of various concentrations of heparin at pH 7.5 and 5.5. Heparin had no effect on VEGF₁₆₅ or VEGF₁₂₁ binding to collagen type I at either pH (FIG. 12). However, low concentrations (1 μg/mL) of heparin potentiated VEGF₁₆₅ binding to fibronectin at pH 7.5, while high concentrations of heparin (100 μg/mL) decreased VEGF₁₆₅ binding. At pH 7.5, heparin had no effect on VEGF₁₂₁ binding to fibronectin, consistent with the fact that VEGF₁₂₁ is unable to interact with heparin at neutral pH. However, at pH 5.5, low concentrations of heparin (1 μg/mL) increased both VEGF₁₆₅ and VEGF₁₂₁ binding to fibronectin, while high concentrations of heparin (100 μg/mL) decreased binding (FIG. 12B). These effects ate consistent with the effects of heparin on VEGF binding to endothelial cells (10,25). Interestingly, these data support our previous finding that VEGF₁₂₁ is converted from a non-heparin binding growth factor at pH 7.5 to one that binds heparin at acidic pH. Therefore, heparin and heparan sulfate might participate with fibronectin to modulate VEGF₁₆₅ and VEGF₁₂₁ binding to the ECM at acidic pH.

Dissociation of VEGF from Fibronectin

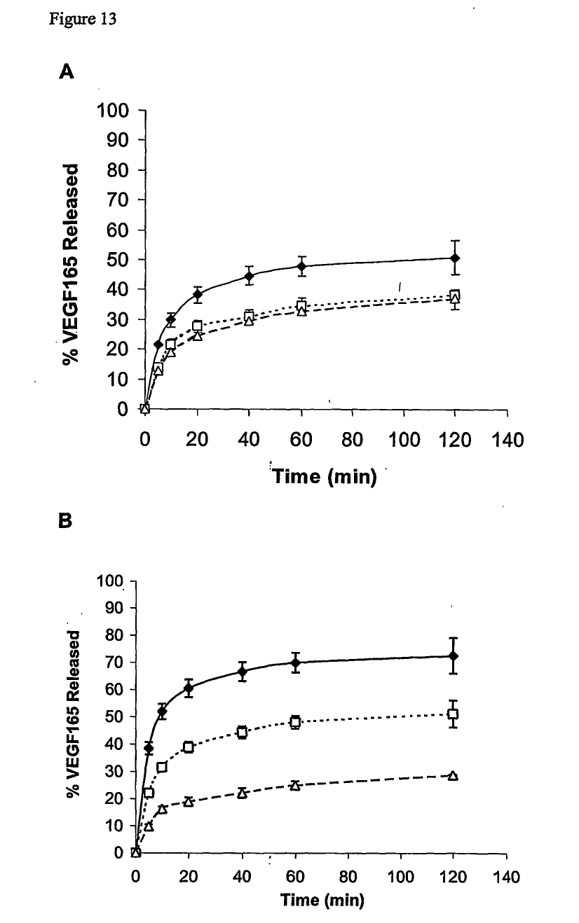

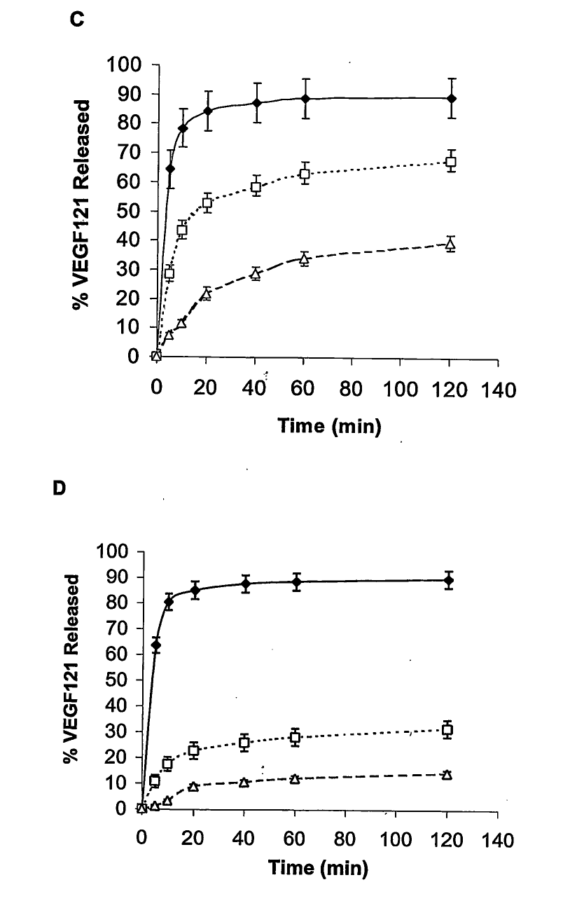

A fibronectin-heparin matrix at acidic pH may be acting to store VEGF₁₆₅ and VEGF₁₂₁ at acidic pH, trapping VEGF at sites of high vascular demand to be released to stimulate endothelial cell invasion. Therefore, we evaluated if VEGF that was bound to fibronectin and fibronectin-heparin matrices at acidic pH, could be stimulated to be released from the matrix by increasing the pH. Experiments were conducted where VEGF₁₆₅ and VEGF₁₂₁ were allowed to bind to fibronectin or fibronectin-heparin dishes at pH 5.5. After binding, unbound VEGF was removed and fresh buffer, adjusted to pH 7.5, 6.5, or 5.5, was added to the wells. Samples were collected at various times and dissociated VEGF was measured. It was found that dissociation of VEGF₁₆₅ and VEGF₁₂₁ from fibronectin and fibronectin-heparin was slow at pH 5.5 compared to pHs 6.5 and 7.5 (FIG. 13). Correspondingly, the calculated rates of VEGF dissociation increased significantly as the pH was increased (Table 2). Consistent with the conditional heparin-binding characteristics of VEGF₁₂₁, heparin caused a significant reduction of VEGF₁₂₁ dissociation at pH 5.5 (FIGS. 13C and D). However, heparin did not reduce VEGF₁₆₅ dissociation at any of the pHs tested. In fact, heparin caused a slight increase in VEGF₁₆₅ release at pH 7.5 suggesting that the VEGF₁₆₅-heparin-fibronectin complexes dissociate faster than the VEGF₁₆₅-fibronectin complexes (FIGS. 13A and B). These results indicate that the presence of the native heparin-binding domain contributes to the overall dissociation of VEGF₁₆₅ from fibronectin-heparin matrices. Thus, low pH stabilizes VEGF-fibronectin complexes, yet these tight interactions can readily be reversed by exposure to neutral pH.

Erk½ Activation by VEGF After Dissociation from Fibronectin

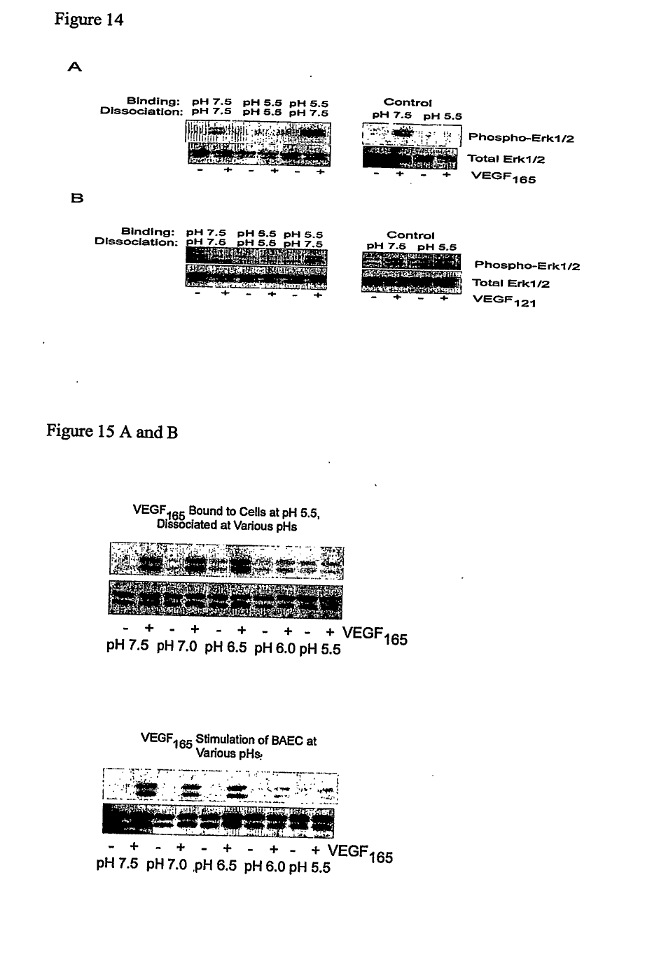

Since we have established that VEGF bound to fibronectin at acidic pH can be released by increasing the pH, we wanted to determine if the dissociated VEGF was active. We used Erk½ activation as a marker for VEGF activity on BAEC. VEGF was bound to fibronectin in the presence of heparin for 1 h at 37° C. at pH 7.5 and pH 5.5. Unbound VEGF was removed and bound VEGF was allowed to dissociate for 20 min in pH 7.5 or pH 5.5 buffer. Dissociated VEGF was then added to quiescent BAEC. Erk½ activation was analyzed after 10 min treatment of the cells. It was found that VEGF₁₆₅ that was bound at pH 7.5 and dissociated at pH 7.5 stimulated a small degree of Erk½ phosphorylation (FIG. 14A). In contrast, VEGF₁₆₅ bound at pH 5:5 and dissociated at pH 5.5 displayed no activation of Erk½. VEGF₁₆₅ bound at pH 5.5 and dissociated from fibronectin at pH 7.5 showed the highest degree of Erk½ phosphorylation. Similar results were observed for VEGF₁₂₁ binding to fibronectin at pH 5.5 and dissociated at pH 7.5 (FIG. 14B). These results indicate that VEGF bound at pH 5.5 and released at pH 7.5 retains its biological activity. Moreover, the relative activities of the various samples are consistent with the VEGF binding and release data (FIGS. 9-13), Which indicates that samples bound at pH 5.5 and released at pH 7.5 would have the highest levels of VEGF. Thus, fibronectin and heparin may act to store VEGF in the ECM at acidic pH in a dormant state, and once the pH is returned to neutral, VEGF is released from these storage sites to stimulate target cells.

VEGF Activation of Erk½ at pH 7.5 After Pre-Bound to BAEC at pH 5.5

VEGF binding to endothelial cells increased under acidic conditions but this did not translate into increased activity at the level of VEGFR-2 and Erk½ phosphorylation. Thus, the increased binding appears to represent a mechanism whereby VEGF is stored in the ECM where it cannot activate cells. At higher pH the stored VEGF can be released and become available to stimulate cells.

We conducted experiments to determine if VEGF bound to cells at pH 5.5 can be induced to activate Erk½ in endothelial cells when the pH is raised. VEGF₁₆₅ was bound to endothelial cells at pH 5.5 for 1 hr at 37° C. Unbound VEGF was removed and new media was added to cells at pH 7.5, 7.0, 6.5, 6.0 or pH 5.5. Cell lysates were collected at 10 min (FIG. 15). It was observed that VEGF₁₆₅ bound to cells at pH 5.5 could activate Erk½ at pH 7.5, 7.0, and 6.5 but not at pH 6.0 or 5.5. In particular, between pH 6.5-6.0 there appears to be a critical switch in activation potential and or release of bound VEGF. Therefore, VEGF₁₆₅ that bound to BAEC at acidic pH was able to stimulate Erk½ activation once the extracellular pH was increased beginning at pH 6.5 and continuing through pH 7.5.

Angiogenesis is an important process that complex organisms use to provide nutrients to cells that are unable to acquire them under their present conditions. One of the major signals that elicits an angiogenic response is hypoxia. Hypoxia has been shown to upregulate VEGF expression (41). Interestingly, fibronectin has been shown to be highly expressed in several tumors (42-45). In addition, hypoxic conditions have been shown to stabilize fibronectin and increase its secretion from cells (46). Hypoxic conditions also lead to decreases in extracellular pH. We have demonstrated that extracellular pH is an important factor in regulating VEGF interactions with cells and the ECM. In particular, we found that VEGF binding to HS is enhanced as the extracellular pH decreases. However, when cells were treated with heparinase III or a combination of heparinase I, II and III (FIG. 8) to remove HS chains, VEGF binding only decreased by ˜40%, suggesting that there are other VEGF binding components involved at low pH. VEGF interactions with fibronectin also increased with decreased pH. Moreover, the addition of exogenous heparin, which interacts with fibronectin and VEGF, enhanced VEGF₁₆₅ and VEGF₁₂₁ binding to fibronectin at acidic pH. In addition, heparin had no effect on VEGF₁₂₁ binding at neutral pH, supporting the concept that VEGF₁₂₁ is converted into a heparin-binding protein at acidic pH. Thus, the regulation of VEGF binding to fibronectin by pH appears to play an important role in localizing VEGF within the ECM in tissues that are in need of vascularization.

We have confirmed that VEGF₁₆₅ and VEGF₁₂₁ that bound to fibronectin at acidic pH, are capable of being rapidly dissociated by increasing the pH to neutral. In addition, when VEGF₁₆₅ and VEGF₁₂₁ were bound to fibronectin in the presence of heparin at pH 5.5, their rates of dissociation were different. The VEGF₁₂₁-heparin-fibronectin complexes that formed at pH 5.5 dissociated significantly slower than VEGF₁₂₁-fibronectin complexes at pH 5.5 and pH 6.5. This indicates that a tight complex forms between VEGF₁₂₁, heparin, and fibronectin that is not easily dissociated at low pH, but once returned to pH 7.5, this complex is destabilized and dissociates at the same rate as VEGF₁₂₁ in the absence of heparin. In contrast, VEGF₁₆₅-heparin-fibronectin complexes formed at pH 5.5 actually dissociated slightly faster than those formed in the absence of heparin at pH 7.5 and 6.5. At 5.5, the presence of heparin had little effect on VEGF₁₆₅ dissociation. These data indicate that VEGF₁₂₁ and VEGF₁₆₅ interact with heparin and fibronectin in different manners, which would contribute to their overall dissociation rate. While VEGF₁₆₅ interacts with heparin through its well-characterized traditional heparin-binding domain (47), both VEGF₁₆₅ and VEGF₁₂₁ may also bind heparin at low pH via a cryptic site that is only competent to bind heparin under acidic conditions. Thus, heparin binding of VEGF₁₂₁ shows complete dependence on pH, while VEGF₁₆₅ retains the ability to bind heparin at neutral pH via its traditional heparin-binding domain, which is required for full biological activity at neutral pH (16,18). Each of these VEGF-heparin interactions would be governed by its own characteristic binding kinetics such that the overall rate of VEGF release from a fibronectin-heparin matrix would represent a composite of all interactions involved. Taken together, these data indicate that the high level of binding of VEGF to fibronectin-heparin matrices can be rapidly reversed as the pH increases toward neutrality. Interestingly, we found that VEGF that was bound to a fibronectin-heparin matrix at pH 5.5, and dissociated at pH 7.5, was able to stimulate Erk½ activation in endothelial cells. Therefore, VEGF that is bound to matrices at low pH and dissociated at neutral pH retains its biological activity toward endothelial cells.

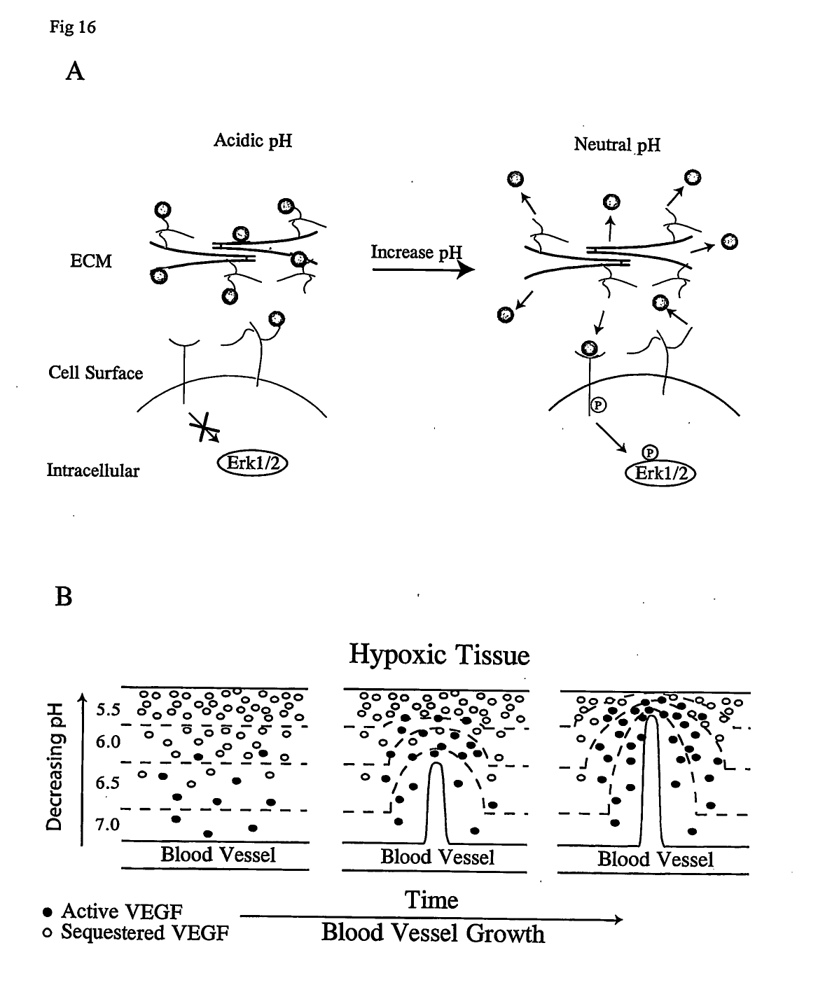

Hypoxia is one of the major inducers of angiogenesis and has been found to upregulate VEGF expression (41,48). In addition, acidic pH upregulates VEGF mRNA in human glioblastoma cells (49). HSPG, which modulate VEGF activity, are upregulated at sites of active angiogenesis, primarily those under hypoxic conditions, such as tumors (19, 21, 50, 51, 52). Also, decreased extracellular pH increases the secretion of fibronectin isoforms in trophoblasts (46). Based on these previous findings and those reported here, we have discovered that hypoxic conditions result in the generation of an acidic extracellular environment, which leads to the storage of VEGF in the extracellular matrix via fibronectin and HSPG (FIG. 16). These acidic locations, by definition, would not be adjacent to the existing vasculature; hence there would be no target endothelial cells in the immediate environment of the secreted VEGF. However, the matrix storage system we discovered indicates the generation of a gradient of VEGF through the reversible binding of VEGF to immobile sites (fibronectin and HSPG) in the matrix (53). While the total amount of VEGF in the ECM would decrease with proximity to existing vessels, the relative proportion of VEGF in an active versus a stored state would be expected to increase. Thus, threshold concentrations of active VEGF could initiate new blood vessel sprouting and growth toward regions containing high VEGF levels. As new vessels move into regions of low pH and high VEGF levels, the corresponding extracellular pH would rise resulting in conversion of stored VEGF to the active form, further stimulating the directed growth and migration of the new vessel. Therefore, a dynamic system of reversible VEGF storage and activation within the ECM could contribute to the positional guidance of new blood vessels to undercirculated/hypoxic/acidic regions of tissue via pH sensitive matrix binding of VEGF.

We have shown that a fibronectin-heparin matrix formulated under acidic pH can have therapeutic applications. For example, a fibronectin-heparin-VEGF matrix would provide a means for the controlled delivery of a pH dependent heparin-binding protein such as VEGF to places where new vasculature is required. Alternatively, using a fibronectin-heparin matrix at acidic pH can also provide a means of depleting VEGF in situations where it is detrimental, such as in certain pathological conditions. While the detailed process by which VEGF regulates angiogenesis remains unclear, the role of local changes in pH in modulating this complex process appears important.

### Example 3

### Preparation of VEGF Encapsulated Spheres

A 1:1 heparin-Sepharose slurry (45 mg/ml, pH 7.5) was combined with fibronectin (0.30 mg/ml), VEGF_{165/121} (1.25 ug/ml), and binding buffer (10 mM HEPES, 1 mg/ml BSA, pH 5.5). The final pH of the mixture was determined to be 5.5. The mixture was incubated for 2 hours at 4° C. with rotation. After incubation, sodium alginate (1.8% w/v, pH 5.5) was added to the solution to produce a final alginate concentration of 1.2% w/v. The solution was gently mixed to ensure uniform distribution. The alginate mixture was then extruded though a 22 gauge needle into cold calcium chloride solution (1.5% w/v, pH 5.5). Calcium alginate spheres were immediately formed upon entering the hardening solution. The alginate spheres were allowed to further crosslink for 5 minutes with gentle mixing, followed by 15 minutes without mixing. The spheres were then washed three times with cold HEPES buffer (10 mM HEPES, pH 5.5). Each sphere had an average diameter of 3-5 mm and contained approximately 0.75 ug heparin, 0.60 ug fibronectin, and 2.0-2.5 ng VEGF. Three spheres were placed in tubes with 2 ml release buffer (10 mM HEPES, 10 mM CaCl₂, 150 mM NaCl, 1 mg/ml BSA) at pH 7.5, 6.5, or 5.5. Tubes were maintained at 4° C. under gentle rotation for the duration of the -experiment. At each time point, 1.5 ml of release buffer was removed from each tube and counted in a gamma counter for quantification of ¹²⁵I-VEGF released. Fresh buffer (1.5 ml) of appropriate pH was added to the tubes at each time point.

In instances when fibronectin was omitted from the spheres, the above procedure was followed with slight exception. The initial concentration of 1:1 heparin-Sepharose slurry was 20 mg/ml and the VEGF concentration was 0.45 ug/ml. Final spheres contained approximately 0.50 ug heparin and 2.2 ng VEGF.

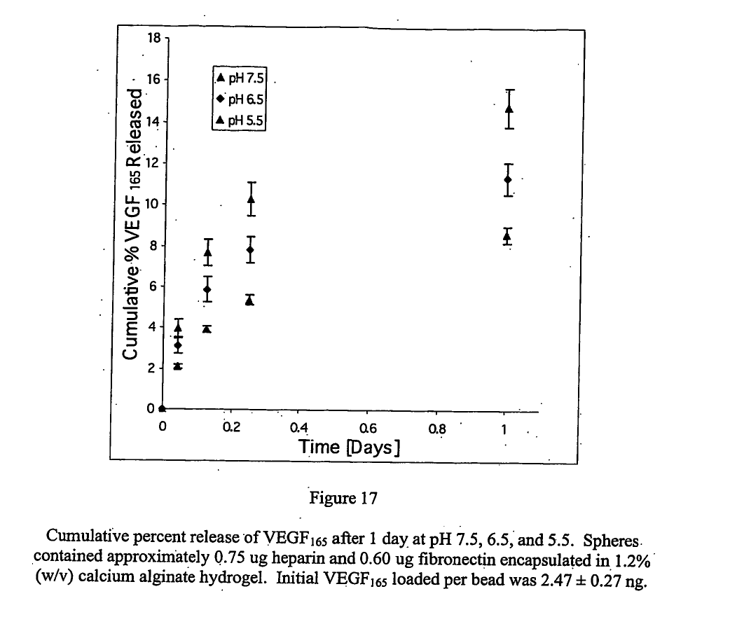

FIG. 17 shows cumulative percent release of VEGF₁₆₅ after 1 day at pH. 7.5, 6.5 and 5.5. Spheres contained approximately 0.75 ug heparin and 0.60 ug fibronectin encapsulated in 1.2% (w/v) calcium alginate hydrogel. Initial VEGF₁₆₅ loaded per bead was 2.47±0.27 ng.

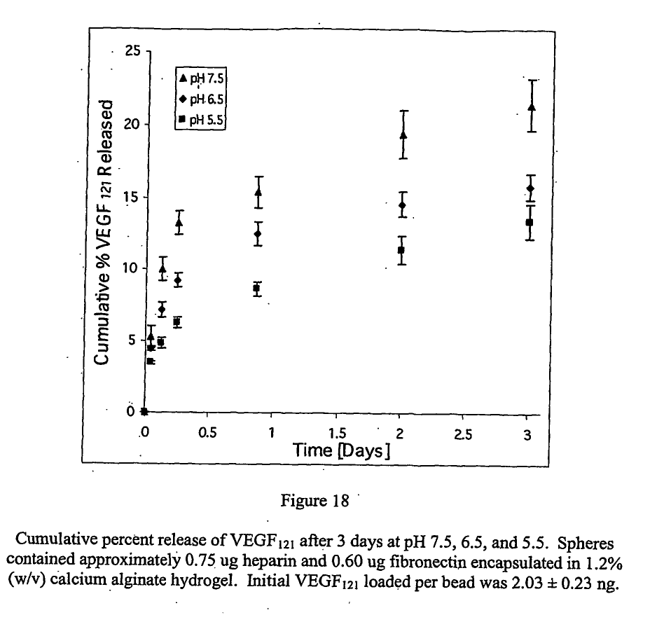

FIG. 18 shows cumulative percent release of VEGF₁₂₁ after 3 days at pH 7.5, 6.5 and 5.5. Spheres contained approximately 0.75 ug heparin and 0.60 ug fibronectin encapsulated in 1.2% (w/v) calcium alginate hydrogel. Initial VEGF₁₂₁ loaded per bead was 2.03±0.23 ng.

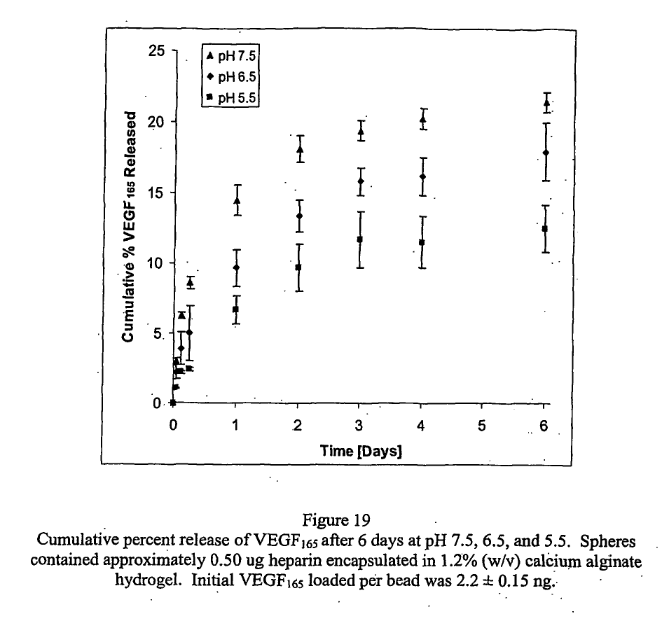

FIG. 19 shows cumulative percent of VEGF₁₆₅ after 6 days at pH 7.5, 6.5 and 5.5. Spheres contained approximately 0.50 ug hepanrn encapsulated in 1.2% (w/v) calcium alginate hydrogel. Initial VEGF₁₆₅ loaded per bead was 2.2±0.15 ng.

TABLE 1
Heparinase III pretreatment to BAEC and endothelial cell deposited
ECM reduced ¹²⁵I-VEGF₁₆₅ and ¹²⁵I-VEGF₁₂₁ binding.

| | VEGF Bound (fmole/10⁵ cells) | | |
|---|---|---|---|
| BAEC | Native | Heparinase III Treated | % Decreased |
| pH 7.5 VEGF₁₆₅ | 0.267 ± 0.007 | 0.103 ± 0.029 | 61.4% |
| pH 5.5 VEGF₁₆₅ | 1.242 ± 0.072 | 0.776 ± 0.043 | 37.6% |
| pH 7.5 VEGF₁₂₁ | 0.037 ± 0.005 | 0.013 ± 0.007 | 64.9% |
| pH 5.5 VEGF₁₂₁ | 0.384 ± 0.018 | 0.254 ± 0.010 | 33.9% |
| Extracellular | VEGF Bound (fmole/well) | | |
| Matrix | Native | Heparinase III Treated | % Decreased |
| pH 5.5 VEGF₁₆₅ | 0.534 ± 0.012 | 0.332 ± 0.024 | 37.8% |
| pH 5.5 VEGF₁₂₁ | 2.194 ± 0.167 | 1.458 ± 0.555 | 33.6% |

TABLE 2
Rates of dissociation of VEGF from fibronectin

| | | VEGF₁₆₅ | | VEGF₁₂₁ | |
|---|---|---|---|---|---|
| | | −Heparin | +Heparin | −Heparin | −Heparin |
| pH 7.5 | k (min⁻¹) | 0.100 | 0.156 | 0.254 | 0.253 |
| | t_{1/2} (min) | 6.9 | 4.4 | 2.7 | 2.7 |
| pH 6.5 | k (min⁻¹) | 0.077 | 0.110 | 0.112 | 0.084 |
| | t_{1/2} (min) | 8.9 | 6.3 | 6.2 | 8.3 |
| pH 5.5 | k (min⁻¹) | 0.047 | 0.046 | 0.034 | 0.004 |
| | t_{1/2} (min) | 14.7 | 15.2 | 20.4 | 181.1 |

VEGF₁₆₅ and VEGF₁₂₁ bound to fibronectin-coated dishes in the presence
(+) and absence (−) of heparin was allowed to dissociate at pH 7.5, 6.5 or
5.5 as described in FIG. 6. The data generated was analyzed using
KaleidaGraph version 3.51 to determine observed dissociation rates based
on a reversible bi-molecular process, as:
${VEGF}_{b} \cdot FN {\to }^{k} {VEGF}_{f} + FN$
where VEGF_{b} · FN represents VEGF bound to fibronectin,
VEGF_{f} is free VEGF, and FN is fibronectin, such that the rate of
dissociation (V) relates to the appearance of free VEGF over times as:
$V = \frac{d [ {VEGF}_{f} ]}{dt} = k [ {VEGF}_{b} ]$
where kᵣ is rate of
$dissociation , {\int }_{{[ {VEGF}_{b} ]}_{0}}^{[ {VEGF}_{b} ]} \frac{d [ [ {VEGF}_{b} ]}{[ {VEGF}_{b} ]} = - {k}_{r} {\int }_{0}^{t} ⅆ t ; [ {VEGF}_{b} ] = {[ {VEGF}_{b} ]}_{0} {ⅇ}^{- {k}_{r} l}$

All references described herein are incorporated herein by reference.

### REFERENCES

1. Risau, W. (1997) Nature 386, 671-674
2. Veikkola, T., Karkkainen, M., Claesson-Welsh, L., and Alitalo, K. (2000) Cancer Res 60, 203-212
3. Robinson, C. J., and Stringer, S. E. (2001) J Cell Sci 114, 853-865
4. Park, J. E., Keller, G. A., and Ferrara, N. (1993) Mol Biol Cell 4, 1317-1326
5. Bernfield, M., Gotte, M., Park, P. W., Reizes, O., Fitzgerald, M. L., Lincecum, J., and Zako, M. (1999) Annu Rev Biochem 68, 729-777
6. Wight, T. N., Kinsella, M. G., and Qwarnstrom, E. E. (1992) Curr Opin Cell Biol 4, 793-801
7. Woods, A., Oh, E. S., and Couchman, J. R. (1998) Matrix Biol 17, 477-483
8. Park, P. W., Reizes, O., and Bernfield, M. (2000) J Biol Chem 275, 29923-29926
9. Sasisekharan, R., Ernst, S., and Venkataraman, G. (1997) Angiogenesis 1, 45-54
10. Turnbull, J., Powell, A., and Guimond, S. (2001) Trends Cell Biol 11, 75-82
11. Esko, J. D., and Lindahl, U. (2001) J Clin Invest 108, 169-173
12. Nugent, M. A., and Iozzo, R. V. (2000) Int J Biochem Cell Biol 32, 115-120
13. Yayon, A., Klagsbrun, M., Esko, J. D., Leder, P., and Ornitz, D. M. (1991) Cell 64, 841-848
14. Rapraeger, A. C., Krufka, A., and Olwin, B. B. (1991) Science 252, 1705-1708
15. Fannon, M., Forsten, K. E., and Nugent, M. A. (2000) Biochemistry 39, 1434-1445
16. Gitay-Goren, H., Soker, S., Vlodavsky, I., and Neufeld, G. (1992) J Biol Chem 267, 6093-6098
17. Tessler, S., Rockwell, P., Hicklin, D., Cohen, T., Levi, B. Z., Witte, L., Lemiscbka, I. R., and Neufeld, G. (1994) J Biol Chem 269, 12456-12461
18. GengrinQvitch, S., Berman, B., David, G., Witte, L., Neufeld, G., and Ron, D. (1999) J Biol Chem 274, 10816-10822
19. Iozzo, R. V., and San Antonio, J. D. (2001) J Clin Invest 108, 349-355
20. Sharma, B., Handler, M., Eichstetter, I., Whitelock, J. M., Nugent, M. A., and Iozzo, R. V. (1998) J Clin Invest 102, 1599-1608
21. Kleeff, J., Ishiwata, T., Kumbasar, A., Friess, H., Buchler, M. W., Lander, A. D., and Korc, M. (1998) J Clin Invest 102, 1662-1673
22. Folkman, J. (1 995) Nat Med 1, 27-31
23. Tannock, I. F. (1972) Br J Radiol 45, 515-524
24. Shweiki, D., Neeman, M., Itin, A., and Keshet, E. (1995) Proc Natl Acad Sci USA 92, 768-772
25. Brogi, E., Schatteman, G., Wu, T., Kim, E. A., Varticovski, L., Keyt, B., and Isner, J. M. (1996) J Clin Invest 97, 469-476
26. Gerber, H. P., Condorelli, F., Park, J., and Ferrara, N. (1997) J Biol Chem 272, 23659-23667
27. Akimoto, T., Liapis, H., and Hammerman, M. R. (2002) Am J Physiol Regul Iitegr Comp Physiol 283, R487-495
28. Yamagata, M., Hasuda, K., Stamato, T., and Tannock, I. F. (1998) Br J Cancer 77, 1726-1731
29. Wike-Hooley, J. L., Haveman, J., and Reinhold, H. S. (1984) Radiother Oncol 2, 343-366
30. Wike-Hooley, J. L., Van der Zee, J., van Rhoon, G. C., Van den Berg, A. P., and Reinhold, H. S. (1984) Eur J Cancer Clin Oncol 20, 619-623
31. Burbridge, M. F., West, D. C., Atassi, G., and Tucker, G. C. (1999) Angiogenesis 3, 281-288
32. D'Arcangelo, D., Facchiano, F., Barlucchi, L. M., Melillo, G., Illi, B., Testolin, L., Gaetano, C., and Capogrossi, M. C. (2000) Circ Res 86, 312-318
33. Nugent, M. A., and Edelman, E. R. (1992) Biochemistry 31, 8876-8883
34. Moscatelli, D. (1988) J Cell Biol 107, 753-759
35. Keyt, B. A., Berleau, L. T., Nguyen, H. V., Chen, H., Heinsohn, H., Vandlen, R., and Ferrara, N. (1996) J Biol Chem 271, 7788-7795
36. Vlodavsky, I., Folkman, J., Sullivan, R., Fridman, R., Ishai-Michaeli, R., Sasse, J., and Klagsbrun, M. (1987) Proc Natl Acad Sci USA 84, 2292-2296
37. Bashlkin, P., Doctrow, S., Klagsbrun, M., Svahn, C. M., Folkman, J., and Vlodavsky, I. (1989) Biochemistry 28, 1737-1743
38. D'Angelo, G., Struman, I., Martial, J., and Weiner, R.I. (1995) Proc Natl Acad Sci USA 92,6374-6378
39. Forsten, K. E., Akers, R. M., and San Antonio, J. D. (200 1) J Cell Physiol 189, 356-365
40. Wahl, M. L., and Grant, D. S. (2000) Gen Pharmacol 35, 277-285
41 Detmar, M., Brown, L. F., Berse, B., Jackman, R. W., Elicker, B. M., Dvorak, H. F., and Claffey, K. P. (1997) J Invest Dermatol 108, 263-268
42. Arihiro, K., Inai, K., Kurihara, K., Takeda, S., and Kaneko, M. (1993) Acta Pathol Jpn 43, 758-764
43. David, L., Nesland, J. M., Holm, R., and Sobrinho-Simoes, M. (1994) Cancer 73, 518-527
44. Hegele, A., Heidenreich, A., Varga, Z., Von Knobloch, R., Olbert, P., Kropf, J., and Hofinann, R. (2003) Urol Res 30, 363-366
45. Ioachim, E., Charchanti, A., Briasoulis, E., Karavasilis, V., Tsanou, H., Arvanitis, D. L., Agnantis, N. J., and Pavlidis, N. (2002) Eur J Cancer 38, 2362-2370
46. Gaus, G., Demir-Weusten, A. Y., Schmitz, U., Bose, P., Kaufinarn, P., Huppertz, B., and Fraik, H. G. (2002) Acta Histochem 104, 51-63
47. Fairbrother, W. J., Champe, M. A., Christinger, H. W., Keyt, B. A., and Starovasnik, M. A. (1998) Structure 6, 637-648
48. Tuder, R. M., Flook, B. E., and Voelkel, N. F. (1995) J Clin Invest 95, 1798-1807
49. Xu, L., Fukumura, D., and Jain, R. K. (2002) J Biol Chem 277, 11368-11374
50. Nackaerts, K., Verbeken, E., Deneffe, G., Vanderschueren, B., Demedts, M., and David, G. (1997) Int J Cancer 74, 335-345
51. Matsuda, K., Maruyama, H., Guo, F., Kleeff, J., Itakura, J., Matsumoto, Y., Lander, A. D., and Korc, M. (2001) Cancer Res 61, 5562-5569
52. Roskams, T., De Vos, R., David, G., Van Damme, B., and Desmet, V. (1998) J Pathol 185, 290-297
53. Dowd, C. J., Cooney, C. L., and Nugent, M. A. (1999) J Biol Chem 274, 5236-5244

1. A device comprising an extracellular matrix having an internal pH between 4.0 and 6.0, wherein the extracellular matrix contains heparin or a heparin-related compound bound to a protein, wherein the protein has a pH dependent binding to the heparin or the heparin-related compound.

2. The device of claim 1, wherein the internal pH is between 5 and 6.

3. The device of claim 1, wherein the internal pH is about 5.5.

4. The device of claim 1, wherein the bound protein is VEGF.

5. The device of claim 1, wherein the extracellular matrix further comprises fibronectin or a fibronectin fragment that binds to the bound protein.

6. (canceled)

7. The device of claim 1, wherein the heparin-related compound is heparan sulfate or heparan sulfate proteoglycan.

8. The device of claim 1, wherein the bound protein contains a heparin-binding consensus sequence.

9. The device of claim 8, wherein the heparin-binding consensus sequence is XBBBXXBX or XBBXBX, where B is a basic amino acid residue or His and X is any amino acid residue.

10. The device of claim 1, wherein the bound protein contains a glycine-like box, wherein said glycine-like box is from about seven to twelve amino acids and contains at least two Gly residues and two-five basic amino acid residues.

11. The device of claim 1, wherein the glycine-like box is SEQ ID NO:1.

12. The device of claim 11, wherein the bound protein is VEGF₁₂₁ or VEGF₁₆₅.

13. The device of claim 1, wherein the device is formed in situ in a subject.

14. A kit for making the device of claim 1, wherein the kit contains a vial containing heparin or a heparin-related compound and a second vial containing fibronectin or a protein containing a heparin-bind domain.

15. The device of claim 1, wherein the extracellular matrix is attached to or encased within a compound selected from the group consisting of a film, a hydrocolloid, a hydrogel, a foam, a gelatin, a bead, a bandage, and a cellophane.

16. The device of claim 1, wherein the heparin-related compound is a heparin-related oligiosaccharide of 8-16 sugars.

17. A method of stimulating angiogenesis at a clinically relevant site in a mammal, said method comprising administering an effective amount of the device of claim 1 capable of releasing a pharmaceutically effective amount of the bound protein at said clinically relevant site.

18. The method of claim 17, wherein said clinically relevant site is a wound and the bound protein is an angiogenic protein that promotes wound healing.

19. The method of claim 17, where the bound protein is VEGF.

20. The method of claim 19, wherein the VEGF is VEGF₁₂₁ or VEGF₁₆₅.

21. The method of claim 17, wherein the device is administered by injection or surgical placement.

22. The method of claim 17, wherein the device is formed in the mammal in situ.

23. The device of claim 4, wherein the extracellular matrix further comprises fibronectin or a fibronectin fragment that binds to the bound protein.

24. The device of 4, wherein the bound protein contains a heparin-binding consensus sequence.

25. The device of 4, wherein the bound protein contains a glycine-like box, wherein said glycine-like box is from about seven to twelve amino acids and contains at least two Gly residues and two-five basic amino acid residues.

26. The device of 7, wherein the bound protein contains a glycine-like box, wherein said glycine-like box is from about seven to twelve amino acids and contains at least two Gly residues and two-five basic amino acid residues.

27. The device of 4, wherein the device is formed in situ in a subject.

28. The device of 7, wherein the device is formed in situ in a subject.

29. The device of 12, wherein the device is formed in situ in a subject.

30. The method of claim 19, wherein the extracellular matrix further comprises fibronectin or a fibronectin fragment that binds to the bound protein and wherein the heparin-related compound is heparan sulfate or heparan sulfate proteoglycan.
